(19) United States
(12) Patent Application Publication
(10) Pub. No.: US 2023/0194540 A1
(43) Pub. Date: Jun. 22, 2023

(54) DIGITAL MICROFLUIDICS ANALYTICAL TECHNIQUES

(71) Applicant: Baebies, Inc., Durham, NC (US)

(72) Inventors: Michael A. Basmajian, Durham, NC (US); Rainer Ng, Durham, NC (US); Miriam H. Nuffer, Durham, NC (US); Jennifer Elderbroom, Durham, NC (US); Michael Boso, Durham, NC (US); Christopher Roberts, Durham, NC (US); Ramakrishna Sista, Durham, NC (US); Vijay Srinivasan, Durham, NC (US); Allen E. Eckhardt, Durham, NC (US); Vamsee K. Pamula, Durham, NC (US)

(73) Assignee: Baebies, Inc., Durham, NC (US)

(21) Appl. No.: 17/925,340

(22) PCT Filed: May 17, 2021
(86) PCT No.: PCT/US2021/032750

§ 371 Date: Nov. 15, 2022

Related U.S. Application Data
(60) Provisional application No. 63/025,555, filed on May 15, 2020.
(60) Provisional application No. 63/074,575, filed on Sep. 4, 2020.
(60) Provisional application No. 63/090,772, filed on Oct. 13, 2020.
(60) Provisional application No. 63/142,046, filed on Jan. 27, 2021.

(51) Int. Cl. G01N 33/68; G01N 33/72; B01L 3/00

(52) U.S. Cl. CPC G01N 33/6827; G01N 33/72; B01L 3/502792; B01L 2200/16

(57) ABSTRACT

Systems and methods for measuring hemoglobin, G6PD activity, and or bilirubin activity in a sample, including measuring the absorbance of a sample, removing background interfering signals, and quantifying the relevant analyte. Systems and methods for reconstituting a reagent in a droplet actuator. Systems and methods for separating plasma from a whole blood sample on a droplet actuator, including combining a sample droplet with an agglutination reagent droplet and using a novel combination of droplet operations to split the sample into a plasma and an agglutinated red blood cell fraction.

### RELATED APPLICATIONS

This application claims priority to and the benefit of U.S. Provisional Patent App. No. 63/025,555 filed on May 15, 2020; U.S. Provisional Patent App. No. 63/074,575 filed on Sep. 4, 2020; U.S. Provisional Patent App. No. 63/090,772 filed on Oct. 13, 2020, and U.S. Provisional Patent App. No. 63/142,046 filed on Jan. 27, 2021; the disclosures of which are hereby incorporated herein by reference.

### FIELD OF THE INVENTION

The invention relates to techniques for improving accuracy and precision for digital microfluidics analytical techniques.

### BACKGROUND

Droplet actuator technology is used to perform a variety of assay types, such as chemistry assays, immunoassays, and molecular assays; in some cases, on the same platform. Assays are typically performed in single-use, disposable cartridges with droplet actuation capabilities and some or all reagents onboard. A wide range of sample types may be used, such as whole blood, serum, urine, saliva, and meconium (newborn stool). Conducting assays on-cartridge introduces unique issues as well as opportunities for improving upon currently available analytical techniques. There is a need for automated techniques that enable improved accuracy and precision for droplet actuator analyses.

### SUMMARY OF THE INVENTION

The invention provides a method of reconstituting a reagent. The method may include providing a dried reagent on a surface, wherein the dried reagent includes one or more reagent components and a dye or tracer. The method may include contacting the dried reagent with a reconstitution buffer to provide a reconstituted reagent droplet. The method may include measuring the dye or tracer in the reconstituted reagent droplet to produce a dye or tracer measurement. The method may include determining based on the dye or tracer measurement the extent of reconstitution of the dried reagent.

In some cases the dried reagent may include a buffer. In some cases the dried reagent may include a diluent reagent. In some cases the dried reagent may include an enzymatic substrate. In some cases the enzymatic substrate may include NADP+. In some cases the enzymatic substrate may include NADP+ and maleimide. In some cases the enzymatic substrate may include G6P and NADP+. In some cases the dried reagent may include an agglutination reagent. In some cases the agglutination reagent may include a hemagglutinating carbohydrate binding protein. In some cases the agglutination reagent may include a hemagglutinating lectin or isolectin or a potato lectin. In some cases the agglutination reagent may include phytohemagglutinin. In some cases the agglutination reagent may include phytohemagglutinin-E (PHAE).

In certain embodiments, the surface includes a surface of a droplet actuator. In certain embodiments, the surface of the droplet actuator may include a substrate surface in a droplet operations gap of the droplet actuator that may be susceptible to contact by a droplet. In certain embodiments, the surface may include a gap facing surface of a top substrate of the droplet actuator. In certain embodiments, the surface may include a gap facing surface of a bottom substrate of the droplet actuator. In certain embodiments, the surface may include a coating on a substrate surface in the droplet operations gap of the droplet actuator. In certain embodiments, the surface may include a surface projecting into or otherwise facing a droplet operations gap of a droplet actuator.

In some cases, the dye or tracer may not be a molecular label. In some cases, the dye or tracer may include a fluorophore. In some cases, the dye or tracer may include Cy3.

In certain embodiments, the reconstitution buffer may include a buffer that may be substantially isotonic to blood. In certain embodiments, the reconstitution buffer may include a phosphate buffer that may be substantially isotonic to blood. In certain embodiments, the reconstitution buffer may include a Tris buffer that may be substantially isotonic to blood. In certain embodiments, the reconstitution buffer may include a non-isotonic buffer that when combined with a reconstituted reagent may be substantially isotonic to blood.

Measuring the dye or tracer in the reconstituted reagent droplet may include determining the absorbance at a wavelength selected to measure the dye or tracer in the reconstituted reagent droplet and may include producing a dye or tracer measurement based on the absorbance. In some cases measuring the dye or tracer may include determining fluorescence at an excitation/emission wavelength selected based on the dye or tracer in the reconstitute reagent droplet and may include producing a dye or tracer measurement based on the fluorescence.

Providing a reconstituted reagent may include stopping an assay process if the extent of reconstitution of the dried reagent is not within a predetermined range. In certain embodiments, providing a reconstituted reagent may include adjusting an assay measurement based on the extent of reconstitution of the dried reagent. In certain embodiments, providing a reconstituted reagent may include using a dye or tracer measurement to create a dilution factor for a reconstituted reagent droplet that may be further diluted in an assay process to provide a diluted reconstituted reagent. In certain embodiments, providing a reconstituted reagent may include assessing whether the diluted reconstituted reagent is within a predetermined range. In certain embodiments, providing a reconstituted reagent may include stopping an assay process if the diluted reconstituted reagent is not within a predetermined range.

The invention provides a method of measuring hemoglobin in a sample. The method may include providing a sample droplet which may include hemoglobin. The method may include obtaining one or more absorbance measurements of the sample droplet at multiple wavelengths from a hemoglobin spectral range, thereby providing an uncorrected hemoglobin absorbance measurement. The method may include using a computing device to correct one or more absorbance measurements for scatter from non-hemoglobin compounds in the sample droplet, thereby providing a corrected hemoglobin absorbance measurement. The method may include using a computing device to calculate, based on the absorbance measurement, the amount of hemoglobin in the sample.

In some cases, the sample may include non-lysed whole blood. In some cases, the sample may include partially lysed whole blood. In some cases, the sample may include lysed whole blood. In some cases, obtaining absorbance measurements may include determining absorbance of the sample droplet in a spectral range from about 400 nm to about 750 nm. In some cases, obtaining absorbance measurements may include determining absorbance of the sample droplet in a spectral range from about 540 nm to about 750 nm. In some cases, correcting the absorbance measurements for non-hemoglobin compounds may include subtracting the non-hemoglobin scatter absorbance measurement from the hemoglobin absorbance measurement. In some cases, the non-hemoglobin scatter absorbance measurement may be made at greater than about 650 nm. In some cases, the non-hemoglobin scatter absorbance measurement may be made in a spectral range from about 700 nm to about 750 nm. In some cases, the non-hemoglobin scatter absorbance measurement may be made at a wavelength selected to detect scatter from lipids.

Calculating the amount of hemoglobin in the sample may include using a computing device to obtain one or more scatter-corrected absorbance measurements of the sample droplet at a wavelength where hemoglobin absorbs. Calculating the amount of hemoglobin in the sample may include using a computing device to multiply by a sample dilution factor and may include using a computing device to multiply by the molar mass of hemoglobin. Calculating the amount of hemoglobin in the sample may include using a computing device to divide by the optical path length. Path length can be measured by use of dyed filler fluid (e.g., oil blue) or other liquids, such as reagent, diluent, buffer etc., with precisely known absorbance properties. Calculating the amount of hemoglobin in the sample may include using a computing device to divide by the molar extinction coefficient at the wavelength where hemoglobin absorbs.

Calculating the amount of hemoglobin in the sample may include using the hemoglobin absorbance measurement to assess a degree of red blood cell lysis in a sample droplet. In certain embodiments, the hemoglobin absorbance measurement may be used to assess the degree of red blood cell lysis in a sample droplet at two or more steps in an assay process. Calculating the amount of hemoglobin in the sample may include stopping an assay process if the degree of red blood cell lysis in a sample droplet is not within a predetermined range.

The invention provides a method of separating plasma from a whole blood sample on a droplet actuator. The method may include providing a sample droplet which may include whole blood. The method may include combining the sample droplet with an agglutination reagent droplet to provide a combined agglutination droplet including a plasma fraction and a red blood cell fraction. The method may include transporting the agglutination droplet in a consistent direction thereby producing a front section of the droplet lacking the red blood cell fraction. The method may include promptly splitting the transported droplet to provide a plasma sample droplet and an agglutinated red blood cell droplet.

In some cases separation of the plasma sample droplet from the agglutination droplet may be accomplished without the use of beads. In some cases separation of the plasma sample droplet from the agglutination droplet may be accomplished without the use of magnetically responsive beads or magnets. In some cases splitting the transported droplet to provide a plasma sample droplet and an agglutinated red blood cell droplet may be accomplished in a droplet operations gap of a droplet actuator using an electrowetting mediated splitting operation. In some cases the splitting may be accomplished in a droplet operations gap of a droplet actuator using an electrowetting mediated splitting operation without the use of magnetically responsive beads or magnets. In some cases a plasma sample droplet may be provided for an albumin assay. In some cases a plasma sample droplet may be provided for a total bilirubin assay.

The invention provides a method of measuring albumin in a plasma sample. The method may include providing a plasma droplet, wherein the plasma droplet may include a dye or tracer reagent. The method may include measuring the dye or tracer in the plasma droplet to produce a plasma dye or tracer measurement, thereby providing a baseline plasma dilution factor. The method may include diluting the plasma droplet with a diluent to provide a diluted plasma droplet and a waste plasma droplet wherein the diluted plasma droplet and the waste plasma droplet may include the dye or tracer reagent. The method may include measuring the dye or tracer in the diluted plasma droplet to produce a diluted plasma dye or tracer measurement, thereby providing a measurement for assessing the dilution process of the plasma from a whole blood sample. The method may include combining the diluted plasma droplet with a rehydrated albumin reagent droplet to provide an albumin product droplet which may include an albumin-reagent complex. The method may include measuring the absorbance of the albumin-reagent complex in the albumin product droplet, thereby providing a measurement for assessing the amount of albumin in the albumin product droplet. The method may include using a computing device for correcting the albumin-reagent complex absorbance measurement for background interfering signals, thereby providing a background corrected albumin-reagent complex absorbance measurement (A_{ALB}). The method may include using a computing device for calculating, based on the background corrected albumin-reagent complex absorbance measurement (A_{ALB}), the amount of albumin in the diluted plasma sample (C*_{ALB}). The method may include using a computing device for calculating, based on the amount of albumin in the diluted plasma droplet (C*_{ALB}), the amount of albumin in a corresponding whole blood sample.

In certain embodiments, the method of measuring albumin in a plasma sample may include providing a plasma droplet from a whole blood sample which may include the above method of separating plasma from a whole blood sample. In certain embodiments, the dye or tracer used in the method of measuring albumin in a plasma sample may not be a molecular label. In certain embodiments, the dye or tracer may include a fluorophore. In certain embodiments, the dye or tracer may include Cy3.

Measuring the dye or tracer in the plasma droplet to provide a baseline plasma dilution factor (DF_{PHAE}) may include obtaining an absorbance spectra of the plasma droplet over a spectral range of from about 650 nm to about 725 nm. In some cases, measuring the dye or tracer in the plasma droplet to provide a baseline plasma dilution factor (DF_{PHAE}) may include obtaining an absorbance spectra of a reference dye or tracer sample over a spectral range of from about 650 nm to about 725 nm. In some cases, measuring the dye or tracer in the plasma droplet to provide a baseline plasma dilution factor (DF_{PHAE}) may include using a computing device for correcting the plasma droplet absorbance spectra and the reference dye or tracer absorbance spectra for scatter or background absorbance over a spectral range of from about 650 nm to about 750 nm. In some cases, measuring the dye or tracer in the plasma droplet to provide a baseline plasma dilution factor (DF_{PHAE}) may include using a computing device for calculating a scatter-corrected average absorbance spectrum for the plasma droplet and a scatter-corrected average absorbance spectrum for the reference dye or tracer sample. In some cases, measuring the dye or tracer in the plasma droplet to provide a baseline plasma dilution factor (DF_{PHAE}) may include using a computing device for correcting the scatter-corrected average absorbance spectrum for the plasma droplet and the scatter-corrected average absorbance spectrum for the reference dye or tracer sample for hemoglobin absorbance. In some cases, measuring the dye or tracer in the plasma droplet to provide a baseline plasma dilution factor (DF_{PHAE}) may include using a computing device for calculating the baseline plasma dilution factor (DF_{PHAE}) as the ratio of hemoglobin-corrected reference dye or tracer absorbance to hemoglobin-corrected plasma droplet absorbance.

In certain embodiments, the absorbance spectra of the plasma droplet and the absorbance spectra of the reference dye or tracer sample may be obtained two or more times. In certain embodiments, the absorbance spectra of the plasma droplet and the absorbance spectra of the reference dye or tracer sample may be obtained four times. In certain embodiments, an absorbance measurement from about 650 nm to about 725 nm of an oil filler fluid blank may be obtained before and after each spectral measurement of the plasma droplet and the reference dye or tracer sample, thereby providing a measure of background noise. In certain embodiments, the dye or tracer may include Cy3. In certain embodiments, correcting the scatter-corrected average absorbance spectra for hemoglobin absorbance may include subtracting absorbance measurements at about 583 nm from absorbance measurements at about 556 nm, thereby providing an absorbance measurements for Cy3 in the plasma droplet and the reference dye or tracer sample.

In some cases, diluting the plasma droplet includes serially diluting the plasma droplet. In some cases, serially diluting the plasma droplet may include a series of 1:3 dilutions. In some cases, the 1:3 dilution may be performed two times.

Measuring the dye or tracer in the diluted plasma droplet to provide a measurement for assessing the dilution process may include obtaining an absorbance measurement of a plasma droplet from each step in the dilution series. In some embodiments, measuring the dye or tracer in the diluted plasma droplet may include comparing the absorbance measurements from each plasma droplet in the dilution series. In some embodiments, measuring the dye or tracer in the diluted plasma droplet may include using a computing device for calculating, based on the absorbance measurements from each plasma droplet, the actual dilution performed at each step in the dilution process.

In some embodiments, the absorbance measurements of the dye or tracer in the diluted plasma droplets may be obtained at about 556 nm and at about 583 nm. In some embodiments, the absorbance measurements of the diluted plasma droplets may be obtained one time.

In some cases, measuring the dye or tracer in the diluted plasma droplet may include obtaining an absorbance measurement of an oil filler fluid blank before and after the absorbance measurement of the diluted plasma droplet, thereby providing a measure of background noise. In some cases, the absorbance measurement of the oil filler fluid blank may be performed over the wavelengths from about 480 nm to about 775 nm.

In some embodiments, combining the diluted plasma droplet with an albumin reagent droplet may include obtaining a diluted plasma droplet absorbance measurement at a wavelength selected to remove background noise signals prior to combining the diluted plasma droplet with the albumin reagent droplet, thereby providing a background diluted plasma droplet measurement. In some embodiments, combining the diluted plasma droplet with an albumin reagent droplet may include obtaining an albumin reagent droplet absorbance measurement at a wavelength selected to remove background noise signals prior to combining the albumin reagent droplet with the diluted plasma droplet, thereby providing a background albumin reagent droplet measurement.

In some cases, the absorbance measurement to remove background signals may be performed at about 630 nm. In some cases, the albumin reagent droplet may include an albumin-binding dye and a buffer. In some cases, the albumin-binding dye may include a triphenylmethane dye. In some cases, the triphenylmethane dye may include bromocresol green. In some cases, the buffer may include a surfactant, may include a detergent, and may include a cyclodextrin. In some cases, the surfactant may include a zwitterionic surfactant. In some cases, the zwitterionic surfactant may include CHAPS. In some cases, the detergent may include a Brij® surfactant. In some cases, the cyclodextrin may include methyl-beta-cyclodextrin.

Measuring the absorbance of the albumin-reagent complex in the albumin product droplet may include obtaining an absorbance measurement of the albumin-reagent complex at a wavelength selected to detect absorption from the albumin-binding dye. In certain embodiments, measuring the absorbance of the albumin-reagent complex in the albumin product droplet may include obtaining an absorbance measurement of an oil filler fluid blank before and after the absorbance measurement of the albumin-reagent complex, thereby providing a measure of background noise. In certain embodiments, measuring the absorbance of the albumin-reagent complex in the albumin product droplet may include subtracting the oil filler fluid absorbance measurement from the albumin-reagent complex absorbance measurement, thereby providing a noise corrected albumin-reagent complex measurement. In some cases, the absorbance measurements of the albumin-reagent complex and the oil filler fluid blank may be obtained at about 630 nm.

Correcting the albumin-reagent complex absorbance measurement for interfering signals may include multiplying the background diluted plasma droplet measurement by 0.5, thereby providing a dilution corrected diluted plasma droplet measurement. Correcting the albumin-reagent complex absorbance measurement for interfering signals may include multiplying the background albumin reagent droplet measurement by 0.5, thereby providing a dilution corrected background albumin reagent droplet measurement. Correcting the albumin-reagent complex absorbance measurement for interfering signals may include subtracting the dilution corrected background albumin reagent droplet measurement and the dilution corrected diluted plasma droplet measurement from the noise corrected albumin-reagent complex measurement, thereby providing a corrected albumin-reagent complex absorbance measurement (A_{ALB}).

Calculating the amount of albumin in the diluted plasma sample may include providing a data set of known albumin concentrations and corresponding absorbance values, using a computing device for correcting the data set to provide a data set with adjusted known albumin concentrations, using a computing device for performing a linear regression analysis using the corrected data set of known albumin concentrations and their corresponding absorbance values to generate a standard curve, using a computing device to calculate the amount of albumin in the diluted plasma droplet (C*_{ALB}) using the corrected albumin-reagent complex absorbance measurement (A_{ALB}) and the linear regression slope and intercept values of the standard curve.

Correcting the data set may include using a computing device for providing a final albumin dilution factor (DF_{ALB}), for providing a final serial dilution factor and may include dividing the known albumin concentrations in the data set by the product of the final albumin dilution factor and the final serial dilution factor, thereby providing a data set with adjusted known albumin concentrations.

Providing the final albumin dilution factor (DF_{ALB}) may include providing a data set of known albumin sample concentrations and hematocrit concentrations, modeling the data set to determine best fit parameters and using a computing device to calculate the final albumin dilution factor (DF_{ALB}) based on the best fit parameters and the baseline plasma dilution factor (DF_{PHAE}). In some cases, providing a final serial dilution factor may include determining the final dilution.

Calculating the amount of albumin in the corresponding whole blood sample may include using a computing device for multiplying the amount of albumin in the diluted plasma droplet (C*_{ALB}) by the final albumin dilution factor (DF_{ALB}) and thereby providing a plasma albumin concentration. Calculating the amount of albumin in the corresponding whole blood sample may include using a computing device for multiplying the plasma albumin concentration by the final serial dilution factor to correct for the serial dilution of the plasma sample, thereby providing a measurement of the amount of albumin in the whole blood sample.

In some cases, the data set of known albumin concentrations and corresponding absorbance values may be stored on cartridge. In some cases, the linear regression slope and intercept values may be stored on cartridge. In some cases, the stored linear regression slope and intercept values may be accessed via reading a barcode.

In some cases, the assay process may be stopped if the dilution of a plasma droplet is not within a predetermined range. In some cases, the assay process may be stopped if an absorbance measurement of a diluted plasma droplet and the corresponding waste plasma droplet are not within a predetermined range.

In some cases, the assay process may be stopped if the rehydrated albumin reagent is not sufficiently rehydrated. Determining whether the albumin reagent has been sufficiently rehydrated may include measuring the absorbance of the rehydrated albumin reagent at about 510 nm and about 750 nm and determining based on the absorbance measurements whether the albumin reagent has been sufficiently rehydrated.

In some cases, the assay process may be stopped if the rehydrated albumin reagent may be contaminated by a physical contaminant. Determining whether the rehydrated albumin reagent may be contaminated by a physical contaminant may include measuring the absorbance of the rehydrated albumin reagent at about 630 nm and about 750 nm and determining based on the absorbance measurements if the quality of rehydration of the albumin reagent is within a predetermined range.

In some cases, the assay process may be stopped if the rehydrated albumin reagent may be contaminated by a high-protein material. In some cases, determining whether the rehydrated albumin reagent may be contaminated by a high-protein material may include measuring the absorbance of the rehydrated albumin reagent at about 630 nm and about 750 nm prior to combining the albumin reagent droplet with the diluted plasma droplet. In some cases, determining whether the rehydrated albumin reagent may be contaminated by a high-protein material may include comparing the absorbance of the rehydrated albumin reagent to the typical absorbance of the rehydrated albumin reagent at these wavelengths. In some cases, determining whether the rehydrated albumin reagent may be contaminated by a high-protein material may include determining if the absorbance of the rehydrated albumin reagent may be higher relative to the typical absorbance of the rehydrated albumin reagent at these wavelengths. In some cases, determining whether the rehydrated albumin reagent may be contaminated by a high-protein material may include calling the rehydrated albumin reagent as contaminated by a high-protein material if the absorbance of the rehydrated albumin reagent may be higher than the typical absorbance of the rehydrated albumin reagent at these wavelengths. In some cases, determining whether the rehydrated albumin reagent may be contaminated by a high-protein material may include using a computing device to correct the absorbance measurement of the rehydrated albumin reagent to offset background noise by subtracting the absorbance measurement at about 750 nm from the absorbance measurement at about 630 nm.

The invention provides a method of measuring G6PD activity in a sample. The method may include providing a sample droplet which may include whole blood. The method may include combining the sample droplet with a G6PD stabilizing reagent droplet to provide a G6PD stabilized sample droplet. The method may include obtaining an absorbance measurement of hemoglobin in the combined reagent and sample droplet, thereby providing a G6PD stabilized sample droplet measure of red blood cell lysis. The method may include diluting the combined reagent and sample droplet with a diluent to provide a lysed sample droplet. The method may include obtaining an absorbance measurement of hemoglobin in the lysed sample droplet to provide a lysed sample droplet measure of red blood cell lysis and an apparent dilution factor. The method may include combining the lysed sample droplet with a G6PD substrate reagent droplet to provide a G6PD−-substrate reaction droplet. The method may include detecting a reaction product in the G6PD−-substrate reaction droplet to produce a measurement of G6PD activity in the sample. The method may include converting the measure of G6PD activity in the sample to a final assay output of a measure of G6PD activity per grams of hemoglobin (U/g Hb). In some cases, the G6PD stabilizing reagent may include NADP+ and maleimide.

Obtaining an absorbance measurement of hemoglobin to provide a G6PD stabilized sample droplet measure of red blood cell lysis may include obtaining one or more absorbance measurements of the G6PD stabilized sample droplet at multiple wavelengths from a hemoglobin spectral range, thereby providing an uncorrected hemoglobin absorbance measurement. Obtaining an absorbance measurement of hemoglobin to provide a G6PD stabilized sample droplet measure of red blood cell lysis may include using a computing device to correct one or more absorbance measurements for scatter from non-hemoglobin compounds in the G6PD stabilized sample droplet, thereby providing a corrected hemoglobin absorbance measurement and a measure of red blood cell lysis. In some cases, obtaining absorbance measurements may include determining the absorbance of a G6PD stabilized sample droplet in a spectral range from about 540 nm to about 750 nm.

In some cases, correcting one or more absorbance measurements for scatter from non-hemoglobin compounds in the G6PD stabilized sample droplet may include subtracting the hemoglobin absorbance measurement at about 750 nm from the hemoglobin absorbance measurement at about 540 nm.

In some cases, diluting the combined reagent and sample droplet may include serially diluting the combined reagent and sample droplet. In some cases, serially diluting the combined reagent and sample droplet may include a series of 1:3 dilutions. In some cases, the 1:3 dilution may be performed two times.

In certain embodiments, obtaining an absorbance measurement of hemoglobin in the lysed sample droplet may include obtaining one or more absorbance measurements of the lysed sample droplet at multiple wavelengths from a hemoglobin spectral range, thereby providing an uncorrected hemoglobin absorbance measurement. In certain embodiments, obtaining an absorbance measurement of hemoglobin in the lysed sample droplet may include using a computing device to correct one or more absorbance measurements for scatter from non-hemoglobin compounds in the lysed sample droplet to provide a corrected hemoglobin absorbance measurement, thereby providing measure of red blood cell lysis and an apparent dilution factor.

In some cases, obtaining absorbance measurements may include determining absorbance of the lysed sample droplet in a spectral range from about 540 nm to about 750 nm. In some cases, correcting one or more absorbance measurements for scatter from non-hemoglobin compounds in the lysed sample droplet may include subtracting the hemoglobin absorbance measurement at about 750 nm from the hemoglobin absorbance measurement at about 540 nm. In some cases, the assay process may be stopped if the measure of red blood cell lysis of the sample droplet is not within a predetermined range.

In certain embodiments, the measure of the apparent dilution factor may include comparing the absorbance measurement of each lysed sample droplet in the dilution series obtained at 540 nm, thereby providing a measure of apparent dilution. In some cases, the assay process may be stopped if the apparent dilution factor of the sample droplet is not within a predetermined range.

In some cases, the G6PD substrate reagent droplet may include G6P and NADP+. In some cases, detecting the product of the G6PD−-substrate reaction may include obtaining kinetic fluorescence measurements of NADPH in the G6PD−-substrate reaction droplet. In some cases, the kinetic fluorescence measurements of NADPH may be obtained in a fluorescence channel from about 360 nm to about 460 nm. In some cases, the kinetic fluorescence measurements may be obtained at about 10 second intervals. In some cases, obtaining kinetic fluorescence measurements may include performing one or more cycles of kinetic fluorescence measurements, thereby providing a set of kinetic fluorescence measurements. In some cases, the set of kinetic fluorescence measurements may include about 5 cycles or about 10 cycles or about 15 cycles or about 20 cycles or about 25 cycles or more. In some cases, obtaining kinetic fluorescence measurements may be performed in less than about 5 minutes. In some cases, obtaining kinetic fluorescence measurements may be performed in less than about 4 minutes.

In certain embodiments, a sweeper buffer droplet between fluorescence measurements may be used to substantially remove or reduce any remnants of the G6PD−-substrate reaction droplet prior to obtaining a subsequent fluorescence measurement.

In certain embodiments, detecting the product of the G6PD−-substrate reaction to produce the measurement of G6PD activity may include obtaining a fluorescence measurement of an oil filler fluid blank before and after obtaining a fluorescence measurement of the G6PD−-substrate reaction droplet, thereby providing a measure of background noise. In certain embodiments, detecting the product of the G6PD−-substrate reaction to produce the measurement of G6PD activity may include averaging the fluorescence measurements of the oil filler fluid blank obtained before and after the fluorescence measurement of the G6PD−-substrate reaction droplet, thereby providing an average blank relative fluorescence unit (RFU) value. In certain embodiments, detecting the product of the G6PD−-substrate reaction to produce the measurement of G6PD activity may include subtracting the average blank RFU value from the fluorescence measurement of the G6PD−-substrate reaction droplet, thereby providing a list of subtracted RFU data. In certain embodiments, detecting the product of the G6PD−-substrate reaction to produce the measurement of G6PD activity may include plotting the subtracted RFU data vs time. In certain embodiments, detecting the product of the G6PD−-substrate reaction to produce the measurement of G6PD activity may include applying a weighting function to the data to remove outlying data points. In certain embodiments, detecting the product of the G6PD−-substrate reaction to produce the measurement of G6PD activity may include using a computing device for calculating a slope, intercept, and a R2 linear regression parameter, thereby providing a measure of NADPH RFU per minute (NADPH RFU/min). In certain embodiments, detecting the product of the G6PD−-substrate reaction to produce the measurement of G6PD activity may include converting the NADPH RFU/min value to a measure of μM NADPH production units of activity per minute (μM NADPH/min). In certain embodiments, detecting the product of the G6PD−-substrate reaction to produce the measurement of G6PD activity may include converting the measure of NADPH production units of activity to activity units per liter (U/L), thereby providing a measure G6PD activity per liter (G6PD U/L).

In some cases, the weighting function may include subjecting positive residuals to a weighting of (1/residual), thereby minimizing their influence on a best fit line, capping the weighting of very small residuals at a given maximum to prevent infinitely high weights, while maximizing their influence on the best fit line, and subjecting negative residuals to a weighting that may be between (1/residual) and the maximum small-residual weight (inclusive).

In certain embodiments, calculating the fit of the linear regression parameter may be an iterative process, thereby providing the best least absolute residual (LAR) fit. In some cases, the assay process may be stopped if a good regression fit cannot be identified.

In some cases, identifying a good regression fit may include using a computing device for calculating the residuals for each data point from the best-fit curve. In some cases, identifying a good regression fit may include marking as poorly fit those residuals which exceed a flat allowable error. In some cases, identifying a good regression fit may include marking as poorly fit those residuals which exceed a percent-based allowable error. In some cases, identifying a good regression fit may include flagging the fit as bad if the number of poorly fit points exceeds a predetermined value. In some cases, the flat allowable error may be 2RFU. In some cases, the percent-based allowable error may be about 20%.

In certain embodiments, converting the measure of G6PD activity to G6PD activity per grams of hemoglobin may include using a computing device for calculating the amount of hemoglobin in the G6PD−-substrate reaction droplet, thereby providing a measure of the amount of hemoglobin in the diluted reaction droplet. Converting the measure of G6PD activity to G6PD activity per grams of hemoglobin may include using a computing device for multiplying the hemoglobin concentration in the diluted reaction droplet by a final dilution factor, thereby providing a measure of the hemoglobin concentration (g Hb/L) in the whole blood sample. Converting the measure of G6PD activity to G6PD activity per grams of hemoglobin may include using a computing device for dividing the measure of G6PD activity (G6PD U/L) by the measure of the hemoglobin (g Hb/L) in the whole blood sample, thereby providing the measure of G6PD activity per grams of hemoglobin (G6PD U/g Hb).

In some cases, calculating the amount of hemoglobin in the G6PD−-substrate reaction droplet may include providing a sample droplet which may include hemoglobin. The method may include obtaining one or more absorbance measurements of the sample droplet at multiple wavelengths from a hemoglobin spectral range, thereby providing an uncorrected hemoglobin absorbance measurement. The method may include using a computing device to correct one or more absorbance measurements for scatter from non-hemoglobin compounds in the sample droplet, thereby providing a corrected hemoglobin absorbance measurement. The method may include calculating, based on the absorbance measurement, the amount of hemoglobin in the sample. In certain cases, the absorbance value for calculating the amount of hemoglobin may be obtained at about 524 nm. In certain cases, the final dilution factor may be 54x. In some cases, the assay process may be stopped if the amount of hemoglobin is not within a predetermined range.

The invention provides a method of measuring total bilirubin in a plasma sample. The method may include providing a plasma droplet wherein the plasma droplet may not include a dye or tracer reagent. The method may include obtaining absorbance measurement of the plasma droplet over a spectral range, thereby providing a background plasma droplet absorbance measurement. The method may include using a computing device to correct the background plasma droplet absorbance measurement for scatter by non-bilirubin compounds, thereby providing a scatter corrected background plasma droplet absorbance measurement. The method may include contacting a dried total bilirubin reagent with the plasma droplet wherein the plasma droplet rehydrates the total bilirubin reagent to provide a reaction droplet which may include a total bilirubin product. The method may include obtaining absorbance measurements of the total bilirubin product in the reaction droplet over a spectral range, thereby providing an absorbance measurement for assessing the amount of total bilirubin in the reaction droplet. The method may include using a computing device to correct the absorbance measurement of the total bilirubin product for scatter by non-bilirubin compounds, thereby providing a scatter corrected total bilirubin product absorbance measurement. The method may include isolating an absorbance signal at a selected wavelength for the total bilirubin product in the reaction droplet, thereby providing an isolated absorbance measurement of the total bilirubin product in the reaction droplet (A_{AZO}). The method may include using a computing device for calculating, based on the isolated absorbance measurement of the total bilirubin product in the reaction droplet (A_{AZO}), the amount of total bilirubin in the plasma sample (C*_{TBIL}). The method may include using a computing device for calculating based on the amount of total bilirubin in the plasma droplet (C *TBIL) the amount of total bilirubin in a corresponding whole blood sample.

In some cases, providing a plasma droplet from a whole blood sample for measuring total bilirubin may include, on a droplet actuator, providing a sample droplet which may include whole blood, combining the sample droplet with an agglutination reagent droplet to provide a combined agglutination droplet which may include a plasma fraction and a red blood cell fraction, transporting the agglutination droplet in a consistent direction thereby producing a front section of the droplet lacking the red blood cell fraction and promptly splitting the transported droplet to provide a plasma sample droplet and an agglutinated red blood cell droplet.

In some cases, providing a background plasma droplet absorbance measurement may include obtaining an absorbance measurement of the plasma droplet over a spectral range, thereby providing an uncorrected background plasma droplet absorbance measurement. The method may include obtaining an absorbance measurement of an oil filler fluid blank over the same spectral range, thereby providing a measure of background noise. The method may include subtracting the oil filler fluid absorbance measurement from the uncorrected background plasma droplet absorbance measurement, thereby providing a noise corrected background plasma droplet absorbance measurement. In some cases, the absorbance measurement of the plasma droplet and the oil filler fluid blank may be obtained from about 450 nm to about 750 nm. In some cases, the absorbance measurement of the plasma droplet may be obtained once. In some cases, the absorbance measurements of the oil filler fluid blank may be obtained before and after the absorbance measurement of the plasma droplet. In some cases, correcting the background plasma droplet absorbance measurement for scatter by non-bilirubin compounds may include subtracting non-bilirubin scatter absorbance measurements from the background plasma droplet absorbance measurement.

In some cases, the non-bilirubin scatter absorbance measurements may be made over the spectral range from about 450 nm to about 750 nm. In some cases, the non-bilirubin scatter absorbance measurements may be made over the spectral range from about 650 nm to about 725 nm. In some cases, the non-bilirubin scatter absorbance measurements may be made at a wavelength selected to detect scatter from proteins. In some cases, the non-bilirubin scatter absorbance measurements may be made at a wavelength selected to detect scatter from lipids. In some cases, the total bilirubin reagent may include the azobilirubin chemistry reagents, for example 3, 5-Dichlorophenyldiazonium tetrafluoroborate and dyphylline.

Providing an absorbance measurement for assessing the amount of total bilirubin product in the reaction droplet may include obtaining an absorbance measurement of the total bilirubin product in the reaction droplet over a spectral range, thereby providing an uncorrected total bilirubin product absorbance measurement. Providing an absorbance measurement for assessing the amount of total bilirubin product in the reaction droplet may include obtaining an absorbance measurement of an oil filler fluid blank over the same spectral range, thereby providing a measure of background noise. Providing an absorbance measurement for assessing the amount of total bilirubin product in the reaction droplet may include subtracting the oil filler fluid absorbance measurement from the uncorrected total bilirubin product absorbance measurement, thereby providing a noise corrected total bilirubin product absorbance measurement.

In some cases, the absorbance measurement of the total bilirubin product and the oil filler fluid blank may be obtained from about 450 nm to about 750 nm. In some cases, the absorbance measurement of the total bilirubin product in the total bilirubin reaction droplet may be obtained once. In some cases, the absorbance measurements of the oil filler fluid blank may be obtained before and after the absorbance measurement of the total bilirubin product in the reaction droplet. In some cases, correcting the total bilirubin product absorbance measurement for scatter by non-bilirubin compounds may include subtracting non-bilirubin scatter absorbance measurements from the total bilirubin product absorbance measurement. In some cases, the non-bilirubin scatter absorbance measurements may be made over the spectral range from about 450 nm to about 750 nm. In some cases, the non-bilirubin scatter absorbance measurements may be made over the spectral range from about 650 nm to about 725 nm. In some cases, the non-bilirubin scatter absorbance measurements may be made at a wavelength selected to detect scatter from proteins. In some cases, the non-bilirubin scatter absorbance measurements may be made at a wavelength selected to detect scatter from lipids.

Isolating the absorbance signal of the total bilirubin product at a selected wavelength may include subtracting the scatter corrected background plasma droplet absorbance measurement from the scatter corrected total bilirubin product absorbance measurement and thereby providing an isolated absorbance signal for the total bilirubin product (A_{AZO}). In some cases, the isolated absorbance signal may be obtained at about 519 nm.

In certain embodiments, calculating the amount of total bilirubin in the plasma sample (C*_{TBIL}) may include providing a data set of known total bilirubin concentrations and corresponding azobilirubin absorbance values. In certain embodiments, calculating the amount of total bilirubin in the plasma sample (C*_{TBIL}) may include using a computing device to correct the data set to provide a data set with adjusted known total bilirubin concentrations. In certain embodiments, calculating the amount of total bilirubin in the plasma sample (C*_{TBIL}) may include performing a linear regression analysis using the corrected data set of know total bilirubin concentrations and their corresponding absorbance values to generate a standard curve. In certain embodiments, calculating the amount of total bilirubin in the plasma sample (C*_{TBIL}) may include using the total bilirubin product (A_{AZO}) isolated absorbance signal and the linear regression slope and intercept values of the standard curve to calculate the amount of total bilirubin in the plasma droplet (C*_{TBIL}).

In some cases, correcting the data set may include using a computing device for providing a final total bilirubin dilution factor (DF_{TBIL}) and may include dividing the known total bilirubin concentrations in the data set by the final total bilirubin dilution factor, thereby providing a data set with adjusted known total bilirubin concentrations.

In some embodiments, providing the final total bilirubin dilution factor (DF_{TBIL}) may include using a computing device for providing a data set of known total bilirubin sample concentrations and hematocrit concentrations. In some embodiments, providing the final total bilirubin dilution factor (DF_{TBIL}) may include modeling the data set to determine best fit parameters. In some embodiments, providing the final total bilirubin dilution factor (DF_{TBIL}) may include calculating the final total bilirubin dilution factor (DF_{TBIL}) based on the best fit parameters and a baseline plasma dilution factor. In some cases, the baseline plasma dilution factor may include a baseline dilution factor (DF_{PHAE}) obtained for a plasma droplet in an albumin assay.

In some cases, the data set of known total bilirubin concentrations and corresponding azobilirubin absorbance values may be stored on cartridge. In some cases, the linear regression slope and intercept values may be stored on cartridge. In some cases, the stored linear regression slope and intercept values may be accessed via reading a barcode.

In certain embodiments, calculating the amount of total bilirubin in the corresponding whole blood sample may include multiplying the amount of total bilirubin in the plasma droplet (C*_{TBIL}) by the final total bilirubin dilution factor (DF_{TBIL}) and thereby providing a measurement of the amount of albumin in the whole blood sample.

In certain embodiments, an absorbance measurement of the plasma droplet may be obtained at about 750 nm, thereby providing a measure for assessing the quality of the plasma droplet. In some cases, the assay process may be stopped if the absorbance measurement is not within a predetermined range. In some cases, the assay process may be stopped if the rehydrated total bilirubin reagent is not sufficiently rehydrated.

In certain embodiments, determining whether the total bilirubin reagent has been sufficiently rehydrated may include measuring the absorbance of the plasma sample at about 498 nm and at about 590 nm. In certain embodiments, determining whether the total bilirubin reagent has been sufficiently rehydrated may include determining based on the absorbance measurements whether the sample contains substantial total bilirubin. In certain embodiments, determining whether the total bilirubin reagent has been sufficiently rehydrated may include comparing the total bilirubin assay result to the presence of substantial total bilirubin visible in the plasma sample to determine whether the total bilirubin reagent has been sufficiently rehydrated.

In some cases, the assay process may be stopped if the plasma droplet shows indications of hemolysis. In some cases, determining if the plasma droplet shows indications of hemolysis may include obtaining an absorbance measurement obtained at about 577 nm. In some cases, determining if the plasma droplet shows indications of hemolysis may include using a computing device for correcting the absorbance measurement of the plasma drop for scatter, thereby providing a scatter corrected plasma drop absorbance measurement. In some cases, determining if the plasma droplet shows indications of hemolysis may include determining based on the absorbance measurement whether the plasma droplet shows signs of hemolysis.

In some cases, measuring total bilirubin in a plasma sample may include screening for lipemia in the plasma sample. In some cases, screening for lipemia may include obtaining an absorbance measurement of the plasma droplet at about 750 nm, thereby providing a measure for assessing the plasma sample for lipemia.

### BRIEF DESCRIPTION OF THE FIGURES

FIG. 1 illustrates a process for serially diluting a whole blood sample to lyse red blood cells.

FIG. 2 illustrates a weighting function applied to negative and positive residuals.

FIG. 3 illustrates successive iterations of fitting data using a weighted least squares linear regression during the least absolute residual regression analysis.

FIG. 4 illustrates two techniques for fitting the absorbance spectrum of the scatter region. FIG. 4a illustrates a simplified Rayleigh scattering model. FIG. 4b illustrates a power transformation and linear regression model.

FIG. 5 is a flow diagram illustrating one technique for separating red blood cells from plasma using droplet operations.

FIG. 6 illustrates a process for performing an albumin assay according to the invention.

FIG. 7 illustrates a process for performing a total bilirubin assay according to the invention.

### TERMINOLOGY

As used herein, the following terms have the meanings indicated.

“Agglutination reagent” means a reagent that causes agglutination of red blood cells. For example, an agglutination reagent may be a hemagglutinating carbohydrate binding protein. Agglutination reagent may, for example, be a hemagglutinating lectin or isolectin. Lectins are known to cause cells in suspension to clump by specifically binding to appropriate glycoconjugates on the surface of adjacent cells. A variety of commercially available lectins are useful for agglutination of red blood cells. The agglutination reagent may be a phytohaemagglutinin, such as phytohemagglutinin-E (PHAE). The agglutination agent may include a tracer or dye, such as a fluorophore. In one aspect of the invention the tracer or dye is Cy3 dye (available from ThermoFisher Scientific, Inc., Waltham, Mass.).

“Droplet” means a volume of liquid on a droplet actuator that is subject to droplet actuation. For example, a droplet may be at least partially bounded by filler fluid, e.g., completely surrounded by filler fluid or bounded by filler fluid and one or more surfaces of the droplet actuator. Droplets may, for example, be aqueous or non-aqueous or may be mixtures or emulsions including aqueous and non-aqueous components. Droplets may take a wide variety of shapes; examples include generally disc shaped, slug shaped, truncated sphere, ellipsoid, spherical, partially compressed sphere, hemispherical, ovoid, cylindrical, and various shapes may be formed during droplet operations, such as merging or splitting or formed as a result of contact of such shapes with one or more surfaces of a droplet actuator.

“DU” means a droplet unit having a droplet volume of 0.9 μL.

“Droplet Actuator” means a device for manipulating droplets. For examples of droplet actuators, see U.S. Pat. No. 6,911,132, entitled “Apparatus for Manipulating droplets by Electrowetting-Based Techniques,” issued on Jun. 28, 2005 to Pamula et al.; U.S. patent application Ser. No. 11/343,284, entitled “Apparatuses and methods for manipulating droplets on a printed circuit board,” filed on Jan. 30, 2006; U.S. Pat. No. 6,773,566, entitled “Electrostatic actuators for microfluidics and methods for using same,” issued on Aug. 10, 2004 and U.S. Pat. No. 6,565,727, entitled “Actuators for microfluidics without moving parts,” issued on Jan. 24, 2000, both by Shenderov et al.; International Patent Application No. PCT/US2006/047486, entitled “Droplet-Based biochemistry,” filed on Dec. 11, 2006, by Pollack et al.; and International Patent Application No. PCT/US2007/009379, entitled “Droplet manipulation systems,” filed on May 9, 2007, by Pollack et al. In various embodiments, the manipulation of droplets by a droplet actuator may be electrode-mediated, e.g., electrowetting-mediated or dielectrophoresis-mediated. Droplet actuators often have two substrates separated by a small distance to form a “droplet operations gap” wherein droplet operations may be conducted.

“Droplet operation” means any manipulation of a droplet on a droplet actuator. A droplet operation may, for example, include: loading a droplet into the droplet actuator; dispensing one or more droplets from a source of droplets; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; condensing a droplet from a vapor; cooling a droplet; disposing of a droplet; transporting a droplet out of a droplet actuator; other operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. Unless otherwise indicated “splitting” is not intended to imply any particular outcome with respect to size of the resulting droplets (i.e., the size of the resulting droplets can be the same or different) or number of resulting droplets (the number of resulting droplets may be 2, 3, 4, 5 or more). The term “mixing” refers to droplet operations which result in more homogeneous distribution of one or more components within a droplet. In various embodiments, the droplet operations may be electrode mediated, e.g., electrowetting mediated or dielectrophoresis mediated. Droplet operations are commonly conducted in a droplet operations gap.

“Filler fluid” means a fluid associated with a droplet operations surface of a droplet actuator, where the fluid is sufficiently immiscible with a droplet phase to render the droplet phase subject to electrode-mediated droplet operations. A wide variety of filler fluids are known in the art. The filler fluid may, for example, be a liquid. The filler fluid may, for example, be a low-viscosity oil, such as silicon oil. Other examples of filler fluids are provided in International Patent Application No. PCT/US2006/047486, entitled, “droplet-Based Biochemistry,” filed on Dec. 11, 2006; and in International Patent Application No. PCT/US2008/072604, entitled “Use of additives for enhancing droplet actuation,” filed on Aug. 8, 2008. In one aspect of the invention, the oil is polydimethylsiloxane, trimethylsiloxy terminated, 5 cSt. In one aspect of the invention, the oil includes a surfactant, such as sorbitan tristearate, e.g., sorbitan tristearate 65. In one aspect, the invention makes use of filler fluid that is a gas, e.g., in droplet operations in air or another gas.

In the various methods of the invention, where steps are described as preceding or following other steps, or similar language, it will be appreciated that unless specifically excluded, other steps may be inserted between steps by one of skill in the art.

Terms like “preferably,”“commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

“Substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

The terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “include,” “includes,” and “including,” are intended to be non-limiting, such that recitation of items in a list is not to the exclusion of other like items that may be substituted or added to the listed items.

### DETAILED DESCRIPTION OF THE INVENTION

The invention provides automated techniques that enable improved accuracy and precision for digital microfluidics analyses (i.e., analyses performed on a droplet actuator using droplet operations), including for example, techniques relating to assays requiring lysis or removal of red blood cells. Examples of automated assays requiring microliter sized sample input that may be performed using the invention are measuring analytes in blood, including without limitation analytes from blood components, such as analytes present in plasma, serum and/or in blood cells, such as red blood cells. For example, Glucose-6-phosphate dehydrogenase (G6PD) enzyme, albumin and/or bilirubin. The invention provides cartridges, instruments, systems, methods and kits for executing these automated techniques.

Quality Control Measures and Flags

The systems of the invention include a variety of quality-control methods and flags to ensure the integrity of the results. A “flag” may be a message communicated by the system to the user, such as, on a display screen, or via an audible sound. A flag may cause an automatic intervention, e.g., stopping further processing of the assay and/or disposing of assay droplets. A flag may pause further processing of the assay and await user input before proceeding with the assay. The system may provide a user the option of stopping further processing or continuing further processing of the assay following a flag. Various flags are discussed throughout the specification.

Chemistry Assay of Enzymatic Activity Using a Kinetic Fluorescence Measurement

In one aspect of the invention, a chemistry assay may be performed using an automated technique requiring lysis or removal of red blood cells which can be used to determine the activity of an enzyme. A sample including, or potentially including, a target enzyme may be incubated with an enzyme substrate to produce a product. The product may be measured quantitatively or qualitatively or both. In one embodiment, kinetic fluorescence measurements may be used to quantify the rate of production of the product of the enzymatic reaction and to relate the rate of production to enzymatic activity. Various droplet operations steps may be conducted during this measurement process, such as electrowetting mediated droplet operations to dispense, merge, split, mix, agitate, etc., sample and reagent droplets.

Reagents

In various embodiments of the invention, it is useful to spot reagents on a substrate of the droplet actuator and reconstitute the reagents using droplets manipulated (e.g., transported using droplet operations) into contact with the spotted reagents using droplet operations. By “spot” the inventors intend to include without limitation placing a reagent or reagents on a substrate by any means and then drying the reagent or reagents under controlled temperature and humidity—e.g., air drying. By “spot reagents on a substrate” the inventors intend to include without limitation depositing on any surface in the droplet operations gap that is susceptible to contact by a droplet using droplet operations. For example, spotting includes depositing a reagent or reagents on coatings of substrates, such as on a hydrophobic coating on the gap facing surface of the top or bottom substrate, as well as spotting on any surface projecting into or otherwise facing the droplet operations gap.

Examples of reagents that may be spotted on a substrate include buffers, diluent reagents, dyes, enzymatic substrates, and the like. For example, the R1 and R2 reagents described below with respect to the G6PD assay may be spotted on a substrate. As another example, the agglutination reagents described below with respect to the plasma sample preparation aspects of the invention may be spotted on a substrate. As another example, the albumin detection reagent described below with respect to the albumin assay may be spotted on a substrate. As a further example, the total bilirubin detection reagent described below with respect to the total bilirubin assay may be spotted on a substrate.

It should be noted that when reconstituting reagents using droplet operations, it may be useful to pulse and/or shuttle the droplets. “Pulsing” means repeatedly activating and deactivating the underlying electrode (e.g. the electrode located underneath the dried reagents) to cause the incoming droplet to pulse over the dried reagents and thereby cause its contents to mix with the dried reagents. “Shuttling” means transporting the droplet back-and-forth, or around a loop, to cause its contents to mix. The frequency of pulsing or of electrode-to-electrode transport can be varied to improve or maximize mixing as needed for particular droplet contents.

In some cases, the system includes checks or flags for rehydration of dehydrated reagents. For example, in some cases the dehydrated reagent may include a colored dye (e.g., Cy3) that may be detected following rehydration. In one example, the reagent is PHAE. In one example, the absorbance of the dyed PHAE reagent is measured after rehydration, allowing a determination of rehydration success (e.g., for Cy3, 520-590 nm; 530-586 nm; or any other combination that measures Cy3). In another embodiment, the rehydrated reagent is diluted, creating a dilution factor (DF). The absorbance of the dye or a tracer may be used to determine whether the PHAE dilution factor (DF_{PHAE}) is within expected ranges. In one example, the dyed PHAE is added to the analyte which dilutes the analyte. In some cases, DFPHAE is calculated for every run to determine the analyte dilution for albumin and/or bilirubin. If the DFPHAE is outside of expected ranges, a flag may be triggered, e.g., a flag that voids the run. In one aspect of the invention, PHAE or analyte dilution may cause differing amounts of optical scatter in an absorbance reading. In another aspect of the invention, it may be useful to ensure that optical scatter is similar across multiple PHAE droplet operation events. If the scatter is not similar, this may trigger a flag, e.g., a flag that voids the run.

Enzymatic Activity Using Whole Blood

The invention provides techniques for measuring enzyme activity from a blood sample, such as a whole blood sample, especially enzymes contained in red blood cells, such as Glucose-6-phosphate dehydrogenase (G6PD).

In various embodiments, the enzymatic assays may include lysis of red blood cells; measurement of hemoglobin, including measurement and elimination of interfering signals from non-hemoglobin compounds, in addition to the measurement of background fluorescence. Furthermore, in various embodiments the enzymatic assays may include indirectly measuring the enzymatic activity by measuring the absorbance and/or fluorescence of the product of the enzymatic reaction on the substrate.

Sample

Any source of red blood cells may be used. In one aspect of the invention, the sample is whole blood. Samples may be collected and stored under conditions which preserve the integrity of red blood cells. A variety of sample collection kits and techniques are available for this purpose. In one aspect of the invention, the whole blood sample may be divided into drops and sent via droplet operations to be used for various assays.

Serial Dilution, Lysis of Red Blood Cells, and Hemoglobin Measurements

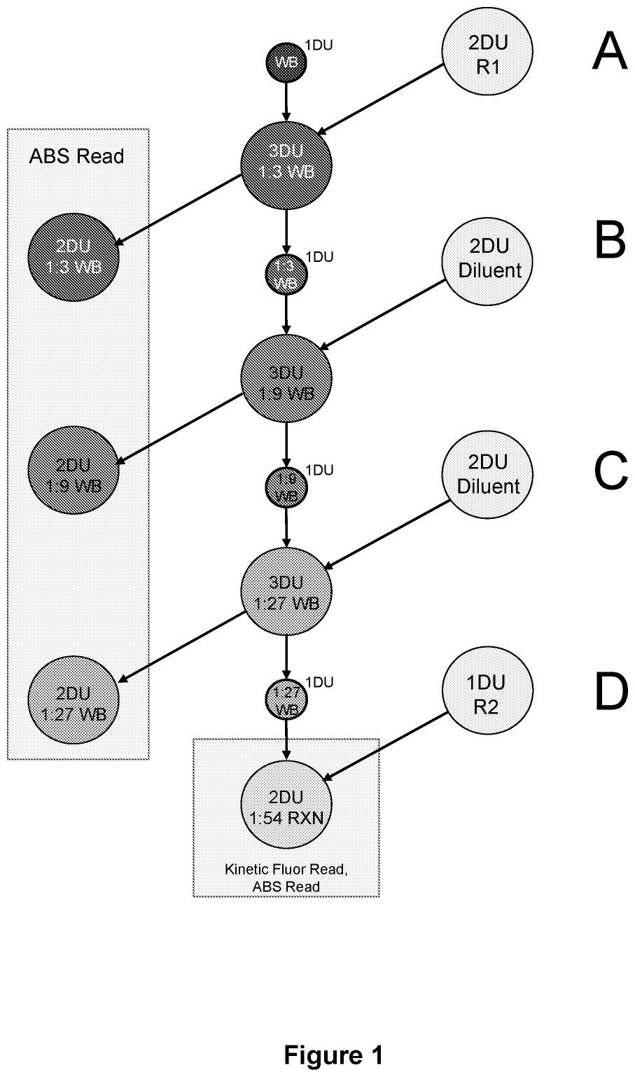

FIG. 1 illustrates a process for serially diluting a whole blood sample to lyse red blood cells. Red blood cells may be lysed osmotically in the cartridge by combining the red blood cell sample, such as whole blood, with a diluent. In one aspect of the invention, the diluent is water. In one aspect of the invention, the assay is a serial dilution assay starting with whole blood where dilution leads to lysis of red blood cells. In this aspect, it can be useful to measure the progressive dilution of the sample and hence the progressive lysis of the red blood cell lysis that occurs. As the assay progresses, and the sample is progressively more diluted, more and more cells are lysed. Measurement of red blood cell lysis can, in one example, be accomplished by measuring the optical scatter of each serially diluted sample. In one embodiment, the optical scatter of hemoglobin can be measured to determine the amount of red blood cell lysis. In one aspect of the invention, optical scatter of diluted samples are measured in the 540-750 nm wavelength range. The hemoglobin spectrum has two characteristic peaks at 540 and 577 nm. These measurements are useful for determining whether the dilution was performed correctly. For example, each step was appropriately ˜3× dilution and the corresponding optical scatter of each diluted sample should decrease by ˜3×. A completely lysed cell should show the absence of optical scatter. Since partially and fully lysed red blood cells release differing amounts of hemoglobin, this check also corresponds to approximate hemoglobin concentration and would also catch a sample that is completely failing to lyse. In one embodiment of the invention, hemoglobin absorbance could be used as a check for completely, incompletely or failed red blood cell lysis. For example, in the G6PD assay described below with reference to FIG. 1, absorbance measurements of hemoglobin concentration are generated at the 1:3 dilution, 1:9 dilution, 1:27 dilution, and 1:54 dilution steps. Of course, this is an example only, and one of skill in the art will appreciate that many alternatives are possible within the scope of the invention.

In certain assays, it may be useful to measure hemoglobin concentration in the red blood cell sample in order to normalize over all samples the amount of enzyme coming from the lysed red blood cells.

Hemoglobin concentration may be measured spectrally using the Beer-Lambert law, e.g., by taking the absorbance between 400 and 600 nm. The hemoglobin spectrum has two characteristic peaks at 540 and 577 nm.

With regard to the measurement of hemoglobin, the inventors have discovered that during detection at certain absorbance measurements relevant to hemoglobin, other compounds such as lipids, cause light scattering, which confounds the hemoglobin signal. Consequently, the inventors have found it useful to correct for scatter by non-hemoglobin compounds in order to achieve a more accurate determination of hemoglobin. By correcting for scatter caused by non-hemoglobin compounds, the methods of the invention help to distinguish hemoglobin absorption from non-hemoglobin scatter, such as scatter from lipids. This aspect of the invention is discussed in further detail below.

Once the red blood cells have been fully lysed, the lysed blood cells may be incubated with an enzyme, the enzyme substrate and other necessary reagents, and the product may be measured. In one embodiment the product may be measured using fluorescence.

Additionally, it is useful to calculate a fluorescence curve that corrects for fluorescence background. For example, cartridge materials or particulates may fluoresce in the desired fluorescence range, e.g., in a 360-460 channel. Correction for fluorescence background may, in one aspect of the invention, be achieved via “blank” fluorescence readings (readings of the cartridge) measured before and after all reacting droplet readings.

Measurement of G6PD Activity Using Whole Blood

In one embodiment of the invention, the enzyme measured can be G6PD. Focusing on the G6PD example illustrated in FIG. 1, in step A, a reagent R1 intended to be combined with whole blood includes β-nicotinamide adenine dinucleotide phosphate (NADP+) and maleimide. For example, R1 may be composed of 5 mM β-nicotinamide adenine dinucleotide phosphate (NADP+), 20 mM maleimide, and methyl-beta-cyclodextrin at 5 mg/mL, in a 20 mM phosphate buffer at a pH of 6.3.

NADP+ helps stabilize the G6PD enzyme during dilution by binding to G6PD in a second, non-enzymatic, “structural” binding site. Maleimide inhibits the NADPH-producing enzyme reaction that follows G6PD in the healthy reaction pathway (specifically 6-phosphogluconate dehydrogenase, or 6PGD). By inhibiting 6PGD, we ensure specificity of the assay. It should be noted that there are other ways to correct for 6PGD, such as in the calculation algorithm (e.g., dividing obtained fluorescence in half). In another embodiment, a 6PGD assay is run in parallel with a no-maleimide G6PD assay and the 6PGD result is subtracted out.

In Step A, a 1DU whole blood droplet WB is combined using droplet operations with a 2DU R1 droplet to produce a 3DU 1:3 diluted whole blood droplet which includes the reagents listed above. The 3DU 1:3 diluted whole blood droplet is then split using droplet operations into one 2DU 1:3 diluted whole blood droplet and a one 1DU 1:3 diluted whole blood droplet. The 2DU 1:3 diluted whole blood droplet is transported using droplet operations into a detection zone for measurement, e.g., measuring the absorbance of the droplet.

In Step B, the 1DU 1:3 whole blood droplet is combined using droplet operations with a 2DU diluent droplet to produce a 3DU 1:9 diluted whole blood droplet. The 3DU 1:9 diluted whole blood droplet is then split using droplet operations into one 2DU 1:9 diluted whole blood droplet and a one 1DU 1:9 diluted whole blood droplet. The 2DU 1:9 diluted whole blood droplet is transported using droplet operations into a detection zone for measuring, e.g., measuring the absorbance of the droplet.

In Step C, the 1DU 1:9 whole blood droplet is combined using droplet operations with a 2DU diluent droplet to produce a 3DU 1:27 diluted whole blood droplet. The 3DU 1:27 diluted whole blood droplet is then split using droplet operations into one 2DU 1:27 diluted whole blood droplet and a one 1DU 1:27 diluted whole blood droplet. The 2DU 1:27 diluted whole blood droplet is transported using droplet operations into a detection zone for measuring, e.g., measuring the absorbance of the droplet.

When whole blood is diluted, such as by the serial dilution in this methodology, osmotic pressure causes the red blood cells to rupture and release some of the contents of theft cells, including hemoglobin, into the surrounding fluid such as blood plasma. Absorbance due to hemoglobin is a direct measurement of the amount of hemoglobin released from the blood cell which then provides a surrogate measurement for the quantity of red blood cells lysed.

At the 1:27 dilution of whole blood, complete hemolysis occurs. At this stage it is expected that the red blood cells are substantially completely ruptured and all of the contents of the cells are released, thus freeing up for measurement the entire amount of G6PD stored in the cells. Additionally, it is helpful to serially dilute the whole blood in order to bring the G6PD concentration into a range measurable by the absorbance or fluorescence detector.

For each of the 2DU diluted waste droplets produced in Steps A, B and C, red blood cell lysis may be quantified as a flag or check by comparing the absorbance of hemoglobin at a wavelength characteristic of one of its peak values (540 nm and 577 nm) to a wavelength characteristic of little to no hemoglobin absorbance while also containing any scatter which may be present throughout the entire wavelength range. In one embodiment of the invention, the absorbance of the 2DU 1:3 waste drop was measured at 540 nm and at 750 nm. The amount of hemoglobin present in the sample and therefore the amount of whole blood hemolysis was determined by subtracting the absorbance of the 2DU 1:3 waste drop at 750 nm from the absorbance of the 2DU 1:3 waste drop at 540 nm. This process was repeated for the 2DU 1:9 waste drop, the 2DU 1:27 waste drop and the 2DU 1:54 sample drop. The measurements may be obtained during steps A, B, and C, such that 2DU 1:3 WB is measured as part of step A, 2DU 1:9 WB is measured as part of step B, and 2DU 1:27 WB is measured as part of step C. Alternatively, any or all of the 2DU droplets may be parked and measured later, e.g., during subsequent steps or thereafter.

Incomplete hemolysis may be confirmed by measuring the amount of hemoglobin relative to the optical scatter at 540 nm and 750 nm for the 2DU 1:3 dilution droplets generated in Step A and the 2DU 1:9 dilution droplets generated in Step B. Complete hemolysis for the droplets having a 1:27 dilution factor may be confirmed by the presence of strong hemoglobin absorption at 540 nm and the absence of all but background (or non-hemoglobin material) scatter at 750 nm in the 2DU 1:27 dilution droplet generated in Step C and the 2DU 1:54 reaction drop generated in Step D.

In step D, the assay is performed using the sample prepared by Steps A-C. The remaining 1DU 1:27 diluted whole blood droplet is combined using droplet operations with a 1DU substrate reagent droplet R2 to produce a 2DU 1:54 reaction droplet.

The substrate reagent droplet includes the relevant enzyme substrate, which in the case of the G6PD assay, may be D-glucose 6-phosphate (G6P) and NADP+. In one example, R2 is composed of 2 mM D-glucose 6-phosphate (G6P), 5 mM NADP+, 2.4 mM MgCl2, and methyl beta cyclodextrin at 5 mg/mL in 100 mM phosphate buffer at a pH of 7.3. The reagents in R2, the reagents of R1 introduced in Step A, and the enzyme G6PD found in the contents of the ruptured red blood cells react to produce 6-phospho-d-glucono-1,5-lactone (6PG) and NADPH, which is the reduced form of NADP+, pursuant to the following reaction (in the presence of G6PD):

D-glucose 6-phosphate (G6P)+NADP⁺□6-phospho-D-glucono-1,5-lactone (6PG)+NADPH+H⁺.

NADPH activity is measured as a surrogate for G6PD activity. The rate of NADPH production is quantified using fluorescence measurements (corresponding to G6PD enzymatic activity) and is proportional to G6PD enzymatic activity. As described above, the reaction occurs in the presence of maleimide, which is used to improve the specificity of the assay by minimizing the production of NADPH from a side reaction of the 6-phosphogluconate dehydrogenase byproduct.

In step D, the 2DU 1:54 reaction droplet is transported into a detection zone of the cartridge for fluorescence measurements. For example, in one aspect of the invention, kinetic fluorescence measurements may be taken. In one aspect of the invention, kinetic fluorescence measurements may be taken by moving the droplet using droplet operations in the presence of a sensor (or into and out of the presence of a sensor) and using the sensor to collect fluorescence measurements. For example, the droplet may be moved by using droplet operations to shuttle the reaction droplet around a loop of electrodes and periodically collecting fluorescence at a designated detection electrode. In one aspect of the invention, the inventors shuttled the reaction droplet along a series of electrodes and back, and collected fluorescence periodically (e.g., every 10 seconds) at a designated detection electrode. This process may be cycled, e.g., 5, 10, 15, 20, 25 or more times. In one aspect of the invention, the process takes less than about 5 minutes or less than about 4 minutes. In various embodiments of the invention, sweeper droplets (buffer) may be transported using droplet operations along the detection path and/or over the detection zone, in order to reduce or remove remnants of the fluorescent droplets that may interfere with subsequent steps in the assay.

The 2DU 1:54 reaction droplet may also be transported into a detection zone of the cartridge for absorbance measurements. For example in one aspect of the invention, spectral absorbance measurements may be taken by moving the droplet using droplet operations in the presence of a sensor (or into and out of the presence of a sensor) and using the sensor to collect absorbance measurements. In one aspect of the invention, hemoglobin present in the lysed droplet is measured through spectral absorbance at a wavelength applicable to hemoglobin. For example, the inventors measured hemoglobin at 524 nm and used the measurement to normalize G6PD enzymatic activity between patients, resulting in a final reported value of G6PD activity per gram of hemoglobin.

In various embodiments of the enzymatic assays described herein, it may be useful to measure enzyme activity over time. For example in the G6PD assay, it may be useful to measure G6PD activity over time. For example, output for the G6PD assay may be measured in activity of G6PD in U/L (U=uM/min of NADPH production). Note that NADPH fluoresces in the 360-460 nm channel.

In one aspect of the invention, it may be useful to include a dilution check in the G6PD assay. G6PD dilution may be monitored by the presence of Hemoglobin (Hb) in the spectral range of 540-750 nm. For example, it may be useful to check the apparent dilution factor upon a G6PD sample going from 1:27 to 1:54, expecting a DF of 2. It may be useful to check the apparent dilution factor upon a G6PD sample going from 1:3 to 1:9, expecting a DF of 3. The 3 to 9 flag may have to be more forgiving due to incomplete hemolysis at this step. It may be useful to check the apparent dilution factor upon a G6PD sample going from 1:9 to 1:27, expecting a DF of 3. It may be useful to check the apparent dilution factor upon a G6PD sample going from 1:3 to 1:27, expecting a DF of 9. In the event of a significant deviation from the expected dilution factor, the flag may indicate that the assay should be stopped.

Analyss of the Data

The values of the measurements described above may be combined to produce units of enzyme activity per gram hemoglobin

$$( \frac{U}{g Hb} ) \qquad ( U / g Hb ) .$$

For the G6PD assay, the values of the measurements described above may be combined to produce units of G6PD activity per gram hemoglobin

$$( \frac{U}{g Hb} ) .$$

For clarity we will first address the fluorescence measurement and how it may be used to generate the value of “units of activity.” Subsequently we will address the absorbance measurement and a general description of how the Beer-Lambert Law is used to determine grams of hemoglobin for the original whole blood sample given that several dilution steps were required to lyse the blood cells in the sample.

Flourescence Calculation

It will be appreciated that a variety of measurement cadences are possible within the scope of the invention. As an example, alternating measurements may be made: blank, sample, blank, sample, repeat . . . For each sample read, the prior blank read (n) may be averaged with the blank read after (n+1). Thus, the average blank relative fluorescence unit (RFU) is an average of the two blank reads bounding a sample read:

$$R F U = \frac{n + ( n + 1 )}{2} .$$

The average RFU of the two bounding blanks before and after a sample can be subtracted to proauce a subtracted RFU data set (Data point=Sample−Averaged Blank). This technique can be repeated for each blank-sample-blank set.

The result is a list of subtracted RFU data. Subtracted RFU can be plotted vs time. For the G6PD assay, this generally produces a positive slope, indicating that the NADPH concentration increases with time in a manner which is proportional to G6PD enzyme concentration. The slope and intercept can be calculated, as well as the R² linear regression (best line fit) parameter. If the fit is good, the system may indicate that the assay is complete.

However, the experience of the inventors has shown that erroneous data spikes are fairly common. As noted above, for example, cartridge components and/or particulates that fluoresce in the 360-460 fluorescence channel can make the data spiky. Spiky data can adversely affect the RFU/time slope calculation. Consequently, it can be useful to reduce the influence of outliers on the calculation.

In one approach to removing outliers, an array of weights is created where the number of elements in the array is the same size as the number of elements in the X and Y data. Initially, all weights can be set to one. In another step of the iterative process, it will be useful to identify which points are close to the best fit line obtained above and which are further away using a R=y₂−y₁ calculation of residuals (R).

The inventors have found that data points with positive differences (those points lying above the fit line) are commonly a problem, and that focusing on reducing the effect of these positive outliers produces good results. Consequently, when applying the weighting function, it is helpful to weight data points that are closer to the best fit line more heavily than data points that are farther away from the line. Further, it is helpful to weight negative residuals more heavily than positive residuals because significant negative outliers are uncommon.

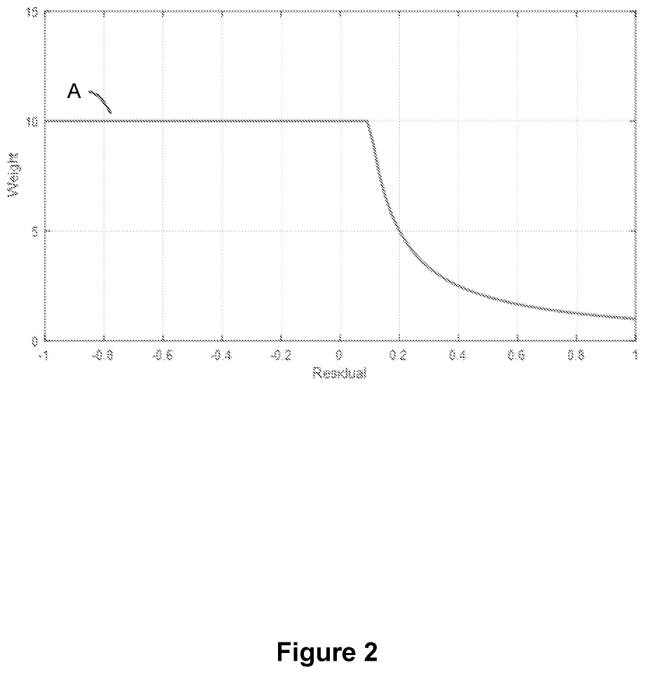

FIG. 2 shows a useful weighting function which applies a capped maximum weight to negative residuals and residuals of data points which fall very close to the best fit line (orange), whereas the curved portion of the line (blue) weights positive residuals less the larger they are (the further the point falls from the best fit line).

To achieve robust linear regression with resistance to positive outliers, the invention makes use of a weighting function. In one aspect, the weighting function meets the following criteria:
1. Larger positive residuals are subject to the blue region of the weighting function which falls between a value of

$$\frac{1}{R}$$

and a maximum small-residual weight (inclusive). The inverse relationship of the magnitude of the larger positive residual to the weight minimizes their influence on the best fit line.
2. The weighting of very small residuals is capped at a given maximum, preventing infinitely high weights, yet still maximizing their influence on the best fit line; and
3. Negative residuals are capped at the same maximum influence as very small residuals; however, these values are found to be rare.

For example, in FIG. 2, the shape of line is defined by:

$$1. Weight = \frac{1}{R\_crit} for R < R\_crit$$

$$2. Weight = \frac{1}{R} for R \geq R\_crit$$

In this illustrative figure, R_crit is 0.1 (hence the shape change at 0.1 producing the maximum weight of 10). R_crit is less than 0.01, or less than 0.001, and preferably is about 0.0001.

It is useful to calculate the least absolute residual (LAR or least absolute value of the data points), which keeps negative outliers and removes the positive outliers. The method produces an array of subtracted RFU data, a corresponding array of weights, and a corresponding array of times, which start at zero and increase. Iterative weighted least-squares regression can be performed, where the weights are inversely proportional to the residuals. Low values and values close to the line are heavily weighted, and high residual outliers are weighted such that the calculation effectively removes them from the fit. It is helpful to iterate this process to find the best LAR fit, i.e., to identify the best specific case of iteratively re-weighted least-squares.

If a good fit cannot be identified, the system may flag the assay for further evaluation, or may reject the assay all together. In one aspect of the invention, a good fit is determined as follows: The residuals for each data point from the best-fit curve are calculated. Residuals which exceed both a flat allowable error (2 RFU) and a percent-based allowable error (20%) are marked as poorly fit. If the number of poorly fit points exceeds 11 (of the 25 total points), the entire fit is regarded as a bad fit and the flag is thrown.

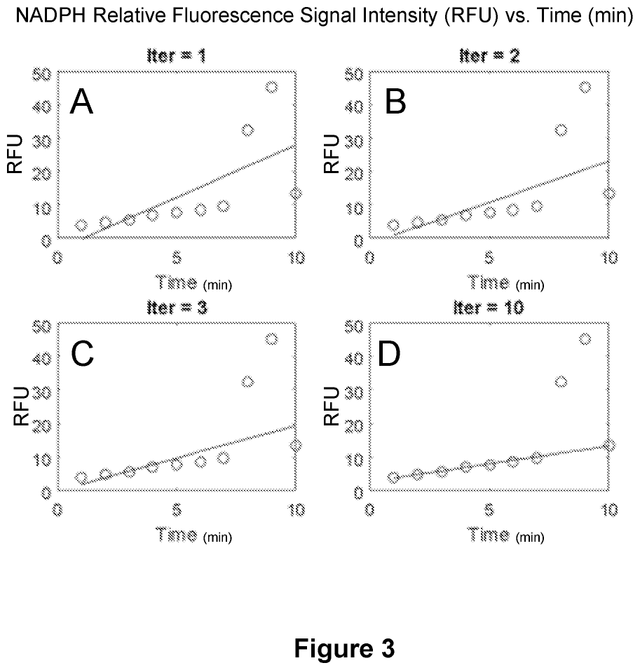

FIG. 3 shows four data plots A, B, C and D, showing time in minutes along the X axis, and signal intensity for fluorescence measurements of NADPH along the Y axis. The plots all show the same “data” which are hypothetical illustrations of typical data based on the inventors' experience with the assay. Each successive panel shows a successive iteration of fitting the same data with a weighted least squares linear regression and this illustrates how several iterations may be used to effectively remove outlier data from the least absolute residual regression analysis. Our experience with the G6PD assay shows that interferences in the assay only produce a positive fluorescence signal and do not produce a lower fluorescing signal. This knowledge allows the inventors to exclude positive outlier data in the least squares linear regression analysis. Panel A illustrates a least squares regression with a poor fit. The two outlier data points (in the upper right corner of panel A) need to be removed. Panels B-C illustrate successive iterations in which positive outliers are weighted less and the regression line, in each panel, more closely fits the relevant data. In the final panel D, the outliers are effectively weighted zero.

When the best fit of the least absolute residuals regression is obtained, the slope is NADPH RFU/minute. As can be seen from FIG. 3, the overall trend of NADPH signal (RFU/minute) generally increases with time. G6PD activity is defined by activity units, where one unit (U) is the amount of enzyme that converts one μmole of NADP+ to NADPH in 1 minute. NADPH RFU/min may be converted via an experimentally determined instrument calibration factor to μM NADPH production units of activity per minute

$$( \frac{μ M NADPH}{min} ) .$$

This is equivalent to the desired unit of U/L (activity units/L) which can be demonstrated using the following conversions:

$$\frac{μ M NADPH}{min} = \frac{μ moles NADPH}{L \cdot min} = \frac{μ moles NADPH / min}{L} = \frac{U}{L} .$$

Using these conversions we obtain G6PD activity per liter (G6PD U/L). The final output

$$\frac{U}{g Hb}$$

is ootainea ay aiviaing G6PD (u/L) by the hemoglobin concentration in grams per liter

$$( \frac{g Hb}{L} )$$

which proceeds via the tollowing conversion steps

$$\frac{U}{L} \div \frac{g Hb}{L} = \frac{U}{L} \times \frac{L}{g Hb} = \frac{U}{g Hb} .$$

We have described how we obtained G6PD activity (U) above. In order to determine grams of hemoglobin per liter of sample, we first calculated the concentration of hemoglobin, which is described below.

It should be noted that hemoglobin (g Hb) levels that are too low (e.g., plasma samples) can lead to unusable G6PD output values. In one aspect of the invention, hemoglobin levels that are too low are at 10 g/L. 10% hematocrit would be roughly 30 g/L. 0% hematocrit (plasma) would be 0 g/L. Hemoglobin levels that are too high (e.g., above 400 g/L, where 300 is roughly 100% hematocrit) may imply that there are other errors present in the assay. Thus, in certain embodiments, the system includes a flag preventing the reporting of the assay value in the event that hemoglobin levels are too low or too high.

Calculating Hemoglobin Concentration

Non-hemoglobin contents of the sample may interfere with attempts to measure hemoglobin. Consequently, the invention may include the use of a scatter correction to correct for interference from non-hemoglobin contents of the sample.

For example, in the G6PD assay, at a wavelength of 524 nm, lipids in the sample elevate the absorbance signal and present as Rayleigh scattering. A scatter correction may be used to remove the elevated signal attributable to lipids at this wavelength. At wavelengths greater than 650 nm, the signal is known from prior information to be completely attributable to Rayleigh scattering of impurities in the sample such as lipids. In order to remove the signal due to Rayleigh scattering from the absorbance peaks of hemoglobin, the signal resulting from scatter in the >650 nm region can be extrapolated to the active hemoglobin spectral range.

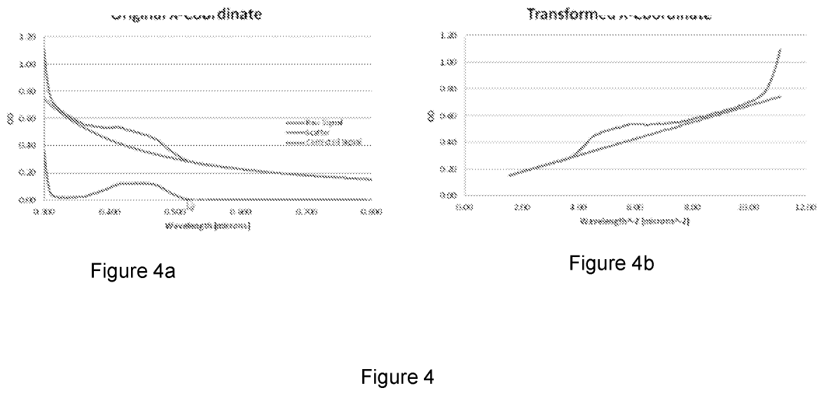

When fitting the absorbance spectrum of the scatter region, the inventors first started with a standard linear regression with a negative slope. However as seen in FIG. 4, the inventors noticed that the trend in the uncorrected absorbance spectrum (the purple curve) has a background signal (the blue curve) which resembles an exponential decay function. This background signal is due to pure Rayleigh and other types of scattering due to interfering substances, such as lipids, in the sample. While heavily rooted in theory, the Rayleigh model is overly sensitive to the curvature in the “pure scatter” region, leading to unreliable extrapolation of the model. Analysis of actual assay data reveals the Rayleigh model frequently overestimates scatter.

To accurately model the scatter in the absorbance signal, we used a simplified model with similar shape to the Rayleigh model: A=aX⁻ⁿ+c, where A is the absorbance, n is an exponent controlling the overall shape of the curve, X is the wavelength, and a and c are fit parameters. The exponent n is chosen before fitting and is optimized for the nature of the scatter to be corrected. For instance, if the scatter were due to insoluble proteins coming out of solution, we set n=1. If scatter were due to the presence of lipids, we set n=2. The values of a and c are found using a least squares regression. While it is possible to model the typical scatter correction signal shown in blue (FIG. 4a), the inventors discovered a simplification. To simplify calculations, one can turn the exponential decay shaped model of scatter correction into a linear function via a power transformation of the X (wavelength) values into Z (wavelength⁻ⁿ) where Z=X⁻ⁿ. As shown in FIG. 4b, the background scatter can now be fit using a linear regression. Finally, the linear regression is reverse transformed back into the wavelength dimension instead of the linear wavelength dimension. The parameters a and c found in a linear regression of A=aZ+c are the same as those for a least squares fit of A=aX⁻ⁿ+c.

We then subtracted this doubly transformed background Rayleigh scattering signal due to large particles in the sample from the sample absorbance spectrum. The resulting spectrum is substantially the absorbance signal attributable to the hemoglobin present in the sample (the red absorbance spectrum in FIG. 4a).

The absorbance of the droplet may be converted to grams of hemoglobin. In one aspect of the invention, the Beer-Lambert law may be used in combination with the droplet volume to convert the absorbance at 524 nm of the droplet to grams of hemoglobin which strongly absorbs at this wavelength. To determine the grams of hemoglobin, for example, the corrected absorbance at 524 nm is multiplied by the sample dilution (27x) and the molar mass of hemoglobin (64500 g/mol), then divided by the optical path length (0.045 cm) and the molar extinction coefficient (29269 L/cmmol). This calculation returns the diluted hemoglobin concentration in the droplet being measured. To determine the concentration of hemoglobin in the original blood sample, the diluted hemoglobin concentration may be multiplied by the 54x dilution factor to obtain the original hemoglobin concentration in the red blood cells in g/L. This final concentration (g Hb/L) represents the denominator of the fraction

$$\frac{U}{L} / \frac{g Hb}{L}$$

which when divided into G6PD activity (U/L) produces the desired value of G6PD activity per gram of hemoglobin,

$$\frac{U}{g Hb} .$$

Error Checking Flags

In some cases it may be helpful to establish methods and flags for colorimetric or fluorimetric assays, such as the G6PD assay. In one example, it may be useful to check the dilution factors, e.g., for the diluted whole blood drops. In one aspect of the invention, the system checks the apparent dilution factor upon a whole blood sample going from about 1:3 to about 1:54. The absorbance readings of the 2DU waste drops may be used to verify the dilution performed on the sample is progressing approximately as anticipated.

In one aspect of the invention, the system may check the absorbance of the 2DU 1:3 waste drop at a particular wavelength. In another aspect of the invention, the system may subtract the background absorbance measured at a particular wavelength. In one example, the system may check the absorbance of the 2DU 1:3 waste drop at 540 nm. In another example, the system may subtract the background absorbance measured at 750 nm from the sample absorbance at 540 nm.

In one embodiment, it may be useful to check the absorbance of the 2DU 1:9 waste drop. In another embodiment, the system may check the absorbance of the 2DU 1:9 waste drop at a particular wavelength. In another aspect of the invention, the system may subtract the background absorbance measured at a particular wavelength. In one example, the system may check the absorbance of the 2DU 1:9 waste drop at 540 nm. In another example, the system may subtract the background absorbance measured at 750 nm from the sample absorbance at 540 nm. In another example the absorbance measured at 540 nm of the 1:3 waste drop may be compared to the absorbance of the 1:9 waste drop to check that an approximately 1:3 dilution has occurred.

In another embodiment, it may be useful to check the absorbance of the 2DU 1:27 waste drop. In one aspect of the invention, the system may check the absorbance of the 2DU 1:27 waste drop at a particular wavelength. In another aspect of the invention, the system may subtract the background absorbance measured at a particular wavelength. In one example, the system may check the absorbance of the 2DU 1:27 waste drop at 540 nm. In another example, the system may subtract the background absorbance measured at 750 nm from the sample absorbance at 540 nm. In one example the absorbance measured at 540 nm of the 1:3 waste drop may be compared to the absorbance of the 1:27 waste drop to check that an approximately 1:9 dilution has occurred. In another example, the absorbance measured at 540 nm of the 1:9 waste drop may be compared to the absorbance of the 1:27 waste drop to check that an approximately 1:3 dilution has occurred.

In another embodiment, it may be useful to check the absorbance of the 2DU 1:54 sample drop. In one aspect of the invention, the system may check the absorbance of the 2DU 1:54 sample drop at a particular wavelength. In another aspect of the invention, the system may subtract the background absorbance measured at a particular wavelength. In one example, the system may check the absorbance of the 2DU 1:54 sample drop at 540 nm. In one example, the system may subtract the background absorbance measured at 750 nm from the sample absorbance at 540 nm. In one example the absorbance measured at 540 nm of the 1:3 waste drop may be compared to the absorbance of the 1:54 sample drop to check that an approximately 1:27 dilution has occurred. In another example, the absorbance measured at 540 nm of the 1:9 waste drop may be compared to the absorbance of the 1:54 sample drop to check that an approximately 1:9 dilution has occurred. In another example, the absorbance measured at 540 nm of the 1:27 waste drop may be compared to the absorbance of the 1:54 sample drop to check that an approximately 1:3 dilution has occurred.

In one embodiment, it may be helpful to check that the hemoglobin concentration in the sample is appropriate for the G6PD calculation. In one example, the system may check for a hemoglobin concentration which is too low, indicating the sample is only plasma or a dilution error occurred. In another example the system may check for hemoglobin concentrations which are too high, indicating a hematocrit concentration which does not make physical sense or a dilution error occurred. The system may check the absorbance of an oil blank before and after the absorbance measurement. In a preferred embodiment, the system may check the absorbance of an oil blank immediately before and immediately after measuring the absorbance of the 2DU 1:27 whole blood waste drop. This embodiment is preferred because any noise in the measurement of the 2DU 1:27 drop translates into noise in the hemoglobin concentration. In another example, the system may measure the absorbance of the 2DU 1:27 whole blood waste drop and then scatter correct this absorbance over a particular wavelength. In another example, the particular wavelength may be 700-750 nm. In another example, the scatter corrected absorbance of the 2DU 1:27 whole blood waste drop at 524 nm may be used in combination with the Beer-Lambert equation to check that the hemoglobin concentration is appropriate for the subsequent calculations.

Separation of Plasma from Whole Blood

In one aspect of the invention plasma is used as the sample for analysis. Plasma may be separated from whole blood using a variety of techniques known in the art, such as centrifugation. In some cases it may be useful to separate plasma from whole blood on the cartridge.

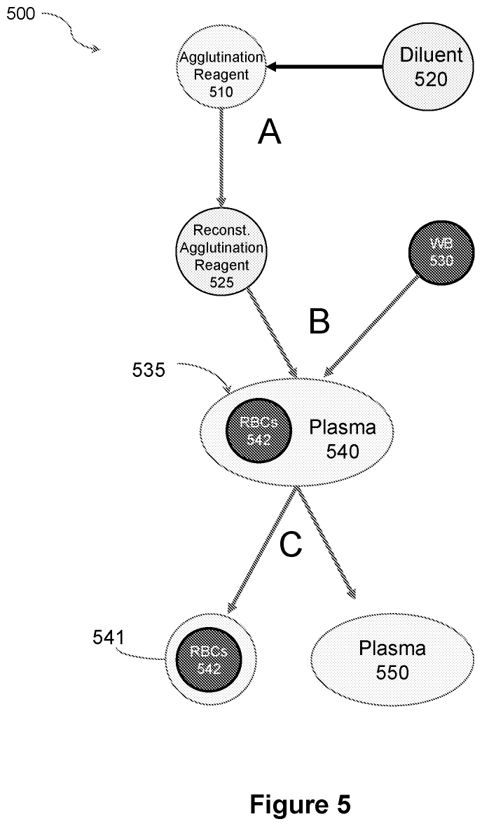

FIG. 5 is a flow diagram illustrating one technique for separating red blood cells from plasma using droplet operations.

The technique makes use of an agglutination reagent 510. The agglutination reagent 510 may be spotted onto a substrate of the cartridge exposed to the droplet operations gap. The spotted agglutination reagent 510 may be air dried under controlled temperature and humidity.

In operation, in Step A, agglutination reagent 510 may be reconstituted using diluent 520. Diluent 520 may be a suitable buffer. Examples of suitable buffers include buffers having osmolarity roughly isotonic to blood. The ions can come from the dry reagent or the rehydration liquid—in the case illustrated here, the dry reagent contains 150 mM NaCl. Other suitable systems include phosphate buffer or tris buffer at appropriate osmolarity. Droplet operations may be used to dispense diluent 520 and transport diluent 520 into contact with agglutination reagent 510 to produce reconstituted agglutination reagent 525.

The process of rehydrating the dried agglutination reagent may involve pulsing the droplet atop the agglutination reagent 510. Pulsing involves repeatedly activating and deactivating the underlying electrode. In some cases, the pulsing may involve at least 10 pulses. In other cases, at least 100 pulses.

A whole blood sample 530 may be provided in the droplet operations gap of the droplet actuator to be combined with the reconstituted agglutination reagent 525. The whole blood sample 530 may be provided using a variety of techniques, such as pipetting, microfluidic pumping, or osmotic pressure. In one aspect of the invention, the whole blood sample 530 is provided by dispensing the whole blood sample from a larger whole blood droplet (not shown) using droplet dispensing operations, such as electrowetting-mediated droplet dispensing operations.

In Step B, reconstituted agglutination reagent 525 and whole blood sample 530 may be merged using droplet operations to yield a combined agglutination droplet 535. The combination results in agglutination of the red blood cells within combined agglutination droplet 535. As the whole blood mixes with reconstituted agglutination reagent 525, the RBCs agglutinate into a clump 542 surrounded by a liquid plasma fraction 540.

The liquid plasma fraction 540 of the droplet 535 may be manipulated by electrowetting in Step C to separate the plasma from the agglutinated RBCs to yield a plasma droplet 550 and leave behind a reduced plasma droplet 541 including the agglutinated red blood cells 542. The inventors have surprisingly found that separation of plasma in this manner does not induce appreciable or substantial hemolysis.

To separate out the plasma from the agglutination droplet 535, it is useful to transport the agglutination droplet 535 in a consistent direction prior to splitting off the plasma droplet 550 so that the frontmost portion of the agglutination droplet 535 will be lacking in agglutinated red blood cells and can be split off to provide a substantially clean plasma droplet 550. As the agglutination droplet 535 with agglutinated red blood cells is transported in a single direction, due to differences in material properties, the agglutinated red blood cells tend to drift to the posterior portion of the droplet, while the anterior portion of the droplet remains substantially free of agglutinated red blood cells. Thus, by reversing transport directions, the invention provides for effective mixing of the agglutination reagent with the whole blood sample, and by maintaining a consistent direction, the invention provides for separation of the agglutinated red blood cells from a portion of the plasma, which can be split off to provide the plasma sample. For example, in the albumin assay performed by the inventors (described below), following the mixing stage in which the direction around an electrode loop was changed, for example, every two times around the loop, the droplet was next transported around the loop in a consistent direction for six consecutive loops prior to splitting off the anterior portion of the droplet to provide a plasma droplet 550. The splitting step was performed using a droplet splitting operation. In some embodiments, the separation of plasma from the agglutination droplet is accomplished without the use of beads. In some embodiments, the separation of plasma from the agglutination droplet is accomplished without the use of magnetically responsive beads or magnets.

Colorimetric, Chemical Plasma Assays

The invention provides methods of testing plasma targets using colorimetric chemical plasma assays. Examples of plasma targets include albumin, total bilirubin, unbound bilirubin, creatinine, potassium, calcium, phosphate, biotin, copper, cobalt, zinc and nickel. Any plasma molecule that can bind to a dye may be a target.

Albumin Assay

As an example of a colorimetric assay, the inventors describe here a test for albumin concentration using bromocresol green (BCG) dye chemistry. The test may be performed on a droplet actuator using droplet operations. Understanding albumin concentration is useful to inform a neonatal hyperbilirubinemia diagnosis and subsequent treatment based on American Academy of Pediatrics guidelines for hyperbilirubinemia. See American Academy of Pediatrics, Subcommittee on Hyperbilirubinemia, “Management of hyperbilirubinemia in the newborn infant 35 or more weeks of gestation,” Pediatrics 2004, 114, 297-316, which is incorporated herein by reference in its entirety.

The albumin assay uses plasma as an input sample. Plasma may be separated from whole blood using a variety of techniques known in the art, such as centrifugation. In some cases it may be useful to first separate plasma from whole blood on the cartridge as described above with respect to FIG. 5 before beginning the colorimetric assay.

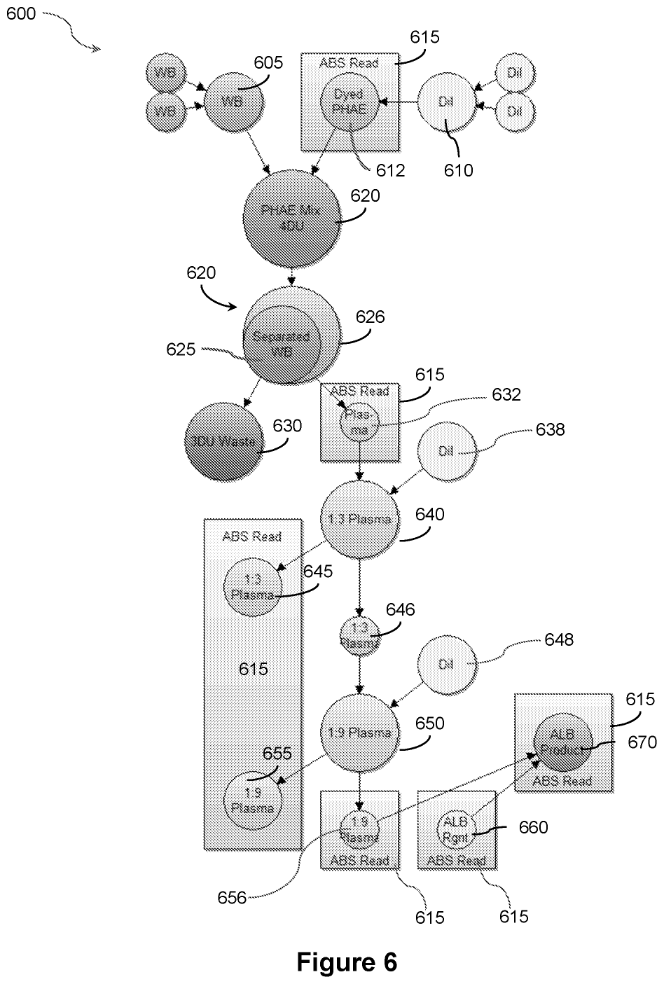

FIG. 6 illustrates a process for performing an albumin assay according to the invention. Whole blood droplets can be dispensed from a whole blood sample (not shown) using droplet operations to provide an initial starting whole blood sample 605. As illustrated here, the whole blood sample 605 is a 2DU droplet.

Diluent droplets can be dispensed from a diluent source using droplet operations to provide an initial starting diluent aliquot droplet 610. As illustrated here, the diluent aliquot droplet 610 is a 2DU droplet.

The 2DU diluent aliquot droplet 610 may be transported using droplet operations into contact with a dried agglutination reagent spot to rehydrate the dried agglutination reagent and provide a reconstituted agglutination reagent droplet 612. In one example, the agglutination reagent is PHAE. In one aspect of the invention the agglutination agent includes a dye or tracer, such as a fluorophore. The dye or tracer is useful to aid in the determination of plasma dilution. In one aspect of the invention, the dye or tracer is Cy3.

The process of rehydrating the dried agglutination reagent may involve pulsing the droplet atop the agglutination reagent. Pulsing involves repeatedly activating and deactivating the underlying electrode. In some cases, the pulsing may involve at least 10 pulses. In other cases, at least 100 pulses.

The reconstituted agglutination reagent droplet 612 may be transported using droplet operations into a detection zone 615 for measuring absorbance of the droplet. In some cases, the absorbance read may be taken multiple times. In one example, the absorbance of an oil blank may be taken before and after the absorbance reads of the droplet. Absorbance of reconstituted agglutination reagent droplet 612 may be measured at a wavelength selected based on the dye included in the reagent, for example. Absorbance measurements will be discussed in detail below.

Whole blood droplet 605 and reconstituted agglutination reagent droplet 612 may be merged using droplet operations to yield an agglutinated droplet 620 to affect agglutination of the red blood cells. Upon agglutination of the red blood cells, the agglutination droplet 620 will separate into plasma 626 and agglutinated red blood cells 625.

Droplet operations may be employed to move the agglutination droplet 620 and thereby cause circulation within the agglutination droplet 620 to improve exposure of the red blood cells to the agglutination reagent. For example, in the albumin assay performed by the applicants, the agglutination droplet 620 is transported using electrowetting-mediated droplet operations around a loop of 18-20 electrodes for at least 4.5 min at 500 ms steps, where the direction is reversed (clockwise/counterclockwise), for example, every 2 loops.

In order to separate out the plasma from the agglutination droplet 620, it is useful to transport the agglutination droplet 620 in a consistent direction prior to splitting off the plasma droplet so that the frontmost portion of the agglutination droplet 620 will be lacking in agglutinated red blood cells and can be split off to provide a substantially clean plasma droplet. As the agglutination droplet 620 with agglutinated red blood cells is transported in a single direction, the agglutinated red blood cells tend to drift to the posterior portion of the droplet, while the anterior portion of the droplet remains substantially free of agglutinated red blood cells. Thus, by reversing transport directions, the invention provides for effective mixing of the agglutination reagent with the whole blood sample, and by maintaining a consistent direction, the invention provides for separation of the agglutinated red blood cells from a portion of the plasma, which can be split off to provide the plasma sample. For example, in the albumin assay performed by the inventors, following the mixing stage in which the direction around an electrode loop was changed, for example, every two times around the loop, the droplet was transported around the loop in a consistent direction for six consecutive loops prior to splitting off the anterior portion of the droplet to provide a 1DU plasma droplet 632 and a 3DU waste droplet 630 composed of the agglutinated red blood cells and a small fraction of unseparated plasma. The splitting step may be performed using a droplet splitting operation.

At this stage in the process, although the volumes of the droplets are known, it is not known by what factor the plasma has been diluted. This is because the red blood cells do not participate in the dilution. Instead the red blood cells are clumped together and aggregate to the side of the 4DU drop. This means the dilution is part plasma and part agglutination reagent. The portion of the dilution due to plasma is variable depending on what percent hematocrit (the ratio of the volume of red blood cells to the total volume of blood) is in the whole blood sample and this varies per patient. Mixing 2DU of whole blood and 2DU of PHAE results in a dilution factor that is a nonlinear function of hematocrit. Without correcting for hematocrit we found 50-200% errors when calculating the dilution of the plasma. Failing to account for the unknown hematocrit concentration on every sample run would produce significant errors. In one aspect of the invention, since the percent hematocrit is unknown in the whole blood sample, a dye was added to the agglutination reagent in order to aid in determining the dilution of the plasma. In one aspect of the invention, spectroscopic techniques may be used to calculate the concentration of the dye and correlate that value to the dilution of the plasma.

It is useful in one aspect of the invention to serially dilute the plasma droplet obtained from the steps described above. In one aspect of the invention, the plasma droplet may be serially diluted to reduce the albumin concentration into a measurable range where absorbance saturation does not occur. It will be understood that a variety of serial dilution processes will be effective within the scope of the invention, however as an example, in the albumin assay performed by the inventors, the 1DU plasma droplet 632 is combined using droplet operations with a 2DU diluent droplet (e.g., water or a buffer) 638 to produce a 3DU 1:3 diluted plasma droplet 640. The 3DU 1:3 diluted plasma droplet 640 is then split using droplet operations into a 2DU 1:3 diluted plasma droplet 645 and a 1DU 1:3 diluted plasma droplet 646. The 2DU 1:3 diluted plasma droplet 645 is transported using droplet operations into a detection zone 615 for an absorbance measurement. The necessity and process of measuring the absorbance of the 1:3 diluted plasma drop 645 will be discussed below.

In one example, the 1DU 1:3 diluted plasma droplet 646 was combined using droplet operations with a 2DU diluent droplet 648 to produce a 3DU 1:9 diluted plasma droplet 650. The 3DU 1:9 diluted plasma droplet 650 was then split using droplet operations into a 2DU 1:9 diluted plasma droplet 655 and a 1DU 1:9 diluted plasma droplet 656, also referred to as the albumin sample drop 656. The 2DU 1:9 diluted plasma droplet 655 was transported using droplet operations into a detection zone 615 for an absorbance measurement. The remaining 1DU 1:9 diluted plasma droplet was separately transported into a detection zone 615 for an absorbance measurement. The necessity and processes of measuring the absorbance of the two separate 1:9 diluted plasma drops 655 and 656 will be discussed below.

In another example, in order to reconstitute the next reagent needed in the assay, a 1DU droplet of diluent (not shown) was dispensed using droplet operations and combined with the spotted albumin detection reagent 660 for at least 2 minutes of pulsing and transport, similar to that described in the separation of plasma above. In that example, separation of two components was desired. In this example, combining of two components is desired and is achieved using droplet operations.

In another step of the assay, the 1DU 1:9 plasma droplet (the albumin sample drop) 656 was combined with a 1DU rehydrated albumin reagent droplet 660. The reaction produces an albumin product drop 670 as discussed below. In one example of the assay, combining these two 1DU droplets in order to produce a one 2DU droplet involves mixing the two 1DU droplets for 45-70 seconds at 350ms per step using droplet operations similar to that described above in the G6PD assay plasma separation step and the albumin detection reagent rehydrating step of this assay (above).

Many different absorbance measurements 615 may be taken throughout the assay to measure the concentration of desired products or as checks to verify the assay is progressing as intended. Absorbance of droplets for which precision is important may be read multiple times, e.g., 2, 3, 4, 5, or more times. However as an example of the albumin assay, absorbance was measured four times each for the 2DU PHAE drop 612 (i.e., a reference dye or tracer sample that comprises the reconstituted agglutination reagent droplet 612 containing only dye and no plasma) and the 1DU dyed plasma droplet 632. In another example of the assay, the absorbance of the diluted dyed plasma drops (2DU 1:3 dyed plasma 645, 1:9 2DU dyed plasma 655, 1:9 1DU dyed plasma 656), the albumin reagent drop 660 and the albumin product drop 670 were each measured once. Furthermore, an absorbance measurement of an oil blank may be collected before and after each set of four sample measurements. In a preferred embodiment for measurements requiring the highest precision, an oil blank may be collected immediately before and immediately after that absorbance measurement. This embodiment is preferred because any noise in the measurement translates into noise in the subsequent calculation. For measurements requiring less precision, the absorbance of an oil blank may be used which was measured at some point before and at some point after the measurement. In one preferred example of the albumin assay, the absorbance of an oil blank was measured immediately before and immediately after the absorbance of each of the 2DU dyed PHAE drop 612, the 1DU dyed plasma drop 632, the 1DU 1:9 albumin sample drop 656, the 1DU rehydrated albumin reagent drop 660, and the 2DU albumin product drop 670. In one preferred step of the assay, an oil blank was measured at some point before and at some point after the absorbance measurement of the 2DU 1:3 and 1:9 dyed plasma waste drops. In one embodiment, each drop may have the background signal removed from its absorbance measurement over a range of wavelengths. In another embodiment, each drop may have the background signal removed from its absorbance measurement at a particular wavelength or both may be used in concert. In one example, the absorbance of the 2DU dyed PHAE drop 612 and the 1DU dyed plasma drop 632 was first scatter corrected as described in the G6PD assay above and then the absorbance of the background hemoglobin signal at 583 nm was subtracted from the absorbance measured at 556 nm. In another example, for the 2DU 1:3 waste drop 645 and the 2DU 1:9 waste drop 655, the absorbance of the background signal at 586 nm was subtracted from the absorbance measured at 530 nm. In another example, for the 1DU albumin sample drop 656, the 1DU albumin reagent drop 660 and the 2DU albumin product drop 670, the absorbance of the background signal at 750 nm was subtracted from the absorbance measured at 630 nm.

The ultimate goal of this assay is to determine the albumin concentration in whole blood using a spectroscopic method. As discussed previously, the albumin is located in the plasma fraction of the whole blood and the plasma can be serially diluted in order to bring the albumin concentration into a measurable range so as not to saturate the instrument. The factor by which the plasma actually is diluted cannot be directly known because of the unknown hematocrit concentration in each sample. In order to determine the amount of dilution in the plasma, a dye may be added to the PHAE agglutination reagent 612. Furthermore, spectroscopic techniques may be used to calculate the dilution of the dye in the dyed PHAE 612 and hence the dilution of the plasma.

In one preferred aspect of the invention, the absorbance spectra 615 of the 2DU dyed PHAE droplet 612 and the 1DU plasma drop 632 were collected four times each over a spectral range of 650 to 725 nm. Next, in one example, each individual spectrum was scatter corrected over the 650 nm to 725 nm spectral range using the method described above with respect to the G6PD assay. Subsequently, the two sets of four scatter corrected absorbance spectra were each averaged together to produce one scatter corrected averaged absorbance spectrum for the 2DU PHAE drop 612 and one scatter corrected averaged absorbance spectrum for the 1DU dyed plasma drop 632.

The scatter corrected, averaged spectra may then be used to establish the ratio of the dye concentration in the undiluted dyed PHAE droplet 612 to the dye concentration in the separated plasma droplet 632, producing the dilution factor of the dye in the PHAE and hence the dilution of the plasma.

In one embodiment of the albumin assay discussed here, the dye may be Cy3. Cy3 dye absorbs in the range of approximately 450 nm to 575 nm, with a peak absorbance of approximately 550 nm. In one embodiment, the wavelengths used for absorbance measurements are 556 nm-583 nm. The 556 nm absorbance measurement 615 was taken near the peak of the Cy3 spectrum, while the 583 nm measurement should show no absorbance from Cy3. Hemoglobin has roughly equal absorption at 556 nm and 583 nm. Therefore, using the difference in absorptivity at these wavelengths one can correct for the contribution of hemoglobin in the Cy3 absorbance spectrum. More specifically, the equation

A_{Cy3}=A_{Cy3@556 nm}−A_{Cy3@583} nm is a measurement sensitive to Cy3 concentration without interference from hemoglobin.

With the ability to determine the absorbance due to the presence of only the dye in the sample, it is now possible to determine the dilution of this dye when the dyed PHAE is mixed with whole blood and therefore determine the dilution of the plasma. In order to determine the dilution factor of the plasma, a ratio may be created of the absorptivity due to only the dye 612 relative to the absorptivity due to the dye diluted by the plasma 632. As discussed above the numerator and denominator of this ratio include the difference in absorption at two separate wavelengths in order to isolate the signal of the dye or tracer from interferences found in the sample. Thus the dilution factor of the plasma may be calculated using the following equation where “612” and “632” represent the scatter corrected, averaged absorptivity values of the 2DU dyed PHAE drop 612 and the scatter corrected averaged absorptivity values of the 1DU plasma drop 632 respectively:

$$D {F}_{PHAE} = \frac{A ( 612 @ 556 nm ) - A ( 612 @ 583 nm )}{A ( 632 @ 556 nm ) - A ( 632 @ 583 nm )} .$$

The numerator is the absorbance of the dyed reconstituted agglutination reagent droplet 612 collected at 583 nm subtracted from the absorbance of the dyed reconstituted agglutination reagent droplet 612 collected at 556 nm. The denominator is the absorbance of the dyed separated plasma droplet 632 collected at 583 nm subtracted from the absorbance of the dyed separated plasma droplet 632 collected at 556 nm. The ratio represents the amount of dilution of the agglutination reagent droplet 612 contents resulting from the combination of whole blood droplet 605 and the reconstituted agglutination reagent droplet 612. The dilution of the dye in the plasma droplet 632 correlates positively with the quantity of plasma in the whole blood sample and negatively with the quantity of red blood cells in the whole blood sample. The dilution factor of the plasma droplet may then be used in calculating the actual dilution performed on the entire sample (as discussed below).

In order to bring the albumin concentration into measurable range, the dyed plasma drop 632 may be serially diluted. In one example, as the 1DU dyed plasma drop 632 is serially diluted, each dilution may produce a 1DU sample drop and a 2DU waste drop. The absorbance 615 of each of these waste drops (the 2DU 1:3 diluted plasma drop 645 and the 2DU 1:9 diluted plasma drop 655) may be used to check that the dilution has occurred properly. These are two of many flags which may be used to check the progress of the assay and will be discussed in detail below. Similarly, the absorbance 615 of the 1DU 1:9 diluted albumin sample drop 656 may be measured as a flag to confirm that the dilution of the sample drop has proceeded as expected and to quantify any background absorbance in the sample such as from lipids.

To calculate the original concentration of the albumin in the whole blood sample 605, the final dilution of albumin may be determined. As an example, the albumin dilution factor (DF_{ALB}) may be calculated using the following experimentally determined equation. To determine the exact albumin sample dilution experienced on the cartridge, a matrix of data was collected from experiments run using samples having different known albumin concentrations and subjecting each sample to varying hematocrit concentrations as well as the agglutination process and 1:9 dilution described with respect to the albumin assay. The resulting plot of experimental results was modeled using a quasi-exponential decay function, where the best fit incorporated the following fit parameters: α=41140, β=6.498 and γ=1.900.

DF_{ALB}=αe^{(-β*BF}^{_{PHAE}})+y

In one aspect of the invention, to determine the concentration of the albumin spectroscopically, the 1:9 diluted albumin sample drop 656 may be combined with a 1DU rehydrated albumin reagent droplet 660 to produce an albumin product droplet 670. The albumin reagent droplet 660 generally may include an albumin-binding dye and a buffer which facilitates the formation of an albumin-dye complex. The absorbance of the albumin-dye complex in the albumin product drop 670 is proportional to its concentration and the concentration of the albumin-dye complex 670 may be directly related to the original albumin concentration in the undiluted whole blood sample 605. This chemical process may be described generally by the following chemical reaction:

Albumin+Dye→Albumin-Dye Complex (“ALB Product” 670)

For example, in the albumin assay illustrated here, the albumin reagent droplet 660 included 1.0 mM Bromocresol Green (BCG, which is in the triphenylmethane dye family), 20 mM CHAPS zwitterionic surfactant (Thermofisher), 0.3% Brij (Thermofisher), and 5 mg/mL methyl-beta-cyclodextrin (Sigma-Aldrich) in 100 mM succinate buffer (pH 3.6). In this example, the reagent facilitates the formation of an albumin-BCG complex 670, where the absorbance of the albumin-BCG complex 670 is proportional to its concentration and the concentration of the albumin-BCG complex may be related back to the original albumin concentration in the undiluted whole blood sample 605.

Albumin+BCG→Albumin-BCG Complex (“ALB Product” 670)

In one aspect of the invention, the absorbance 615 of the albumin-BCG complex 670 was measured at 630 nm with the absorbance of an oil blank measured before and after the absorbance measurement of the albumin-BGC complex 670. In a preferred embodiment, the absorbance of the oil blank was measured immediately before and immediately after the absorbance of the albumin-BCG complex 670. This embodiment is preferred because any noise in the measurement of the albumin-BCG complex 670 translates into noise in the final concentration. It can be understood that subtracting noise in the oil blank from the sample absorbance would remove the influence of any noise present from the final calculation. However, to convert the noise corrected absorbance of the albumin-BCG complex 670 to the concentration of the albumin-BCG complex 670, the absorbance was first corrected for other background interferences, for example. In one aspect, the absorbance 615 of the 1:9 diluted plasma (albumin sample) drop 656 was measured at 630 nm and represents absorption due to background interferences such as lipids in the sample. The absorbance 615 of the albumin reagent drop 660 is measured at 630 nm and represents absorption due to background interferences from unreacted albumin detection reagent. The following equation was used to calculate the background signal corrected absorbance of the albumin-BCG complex 670 (A_{AIB}), where A₆₇₀ is the absorbance of the albumin-BCG complex 670, A₆₆₀ is the absorbance of the albumin reagent 660, and A₆₅₆ is the absorbance of the albumin sample drop 656. The ½ in the formula corrects for the 1:2 dilution of the 1DU albumin sample drop 656 and the 1DU albumin reagent drop 660.

$${A}_{A L B} = {A}_{670} - \frac{1}{2} {A}_{660} - \frac{1}{2} {A}_{656}$$

In one aspect of the invention, the absorbance of the AALB may be converted to pre-corrected albumin concentration using a factory generated standardized curve of pairs of known albumin concentrations and their resulting absorbance values. It may be understood that a linear regression may be experimentally determined in the factory from these data pairs and the linear regression slope and intercept values may be stored on the cartridge via the barcode, which the instrument reads. In one embodiment, AALB values obtained on the cartridge may be multiplied by the stored slope and that product may then be added to the stored intercept to provide the pre-corrected albumin concentration. In one embodiment, when a new albumin reagent is made, this calibration may be performed and the resulting linear regression parameters may be updated and then stored on the cartridge.

In a preferred embodiment, the known albumin concentration in the concentration-absorbance data sets above was first divided by the 9*DF_{ALB} determined for each sample on the cartridge, where “9” is the 1:9 dilution of the albumin sample drop 656. The linear regression was then performed using the following data pairs: [(known albumin concentration/(9*DF_{ALB})), resultant absorbance values] as can be seen in the following equation, where Known [ALB] and Absorbance of Known [ALB] are data pairs generated in the laboratory and DFALB is generated for each sample run.

$$\frac{Known [ ALB ]}{9 \cdot {DF}_{ALB}} = m \cdot Absorbance of Known [ Alb ] + b$$

In this preferred example, the linear regression was run for each sample and the slope and intercept values may be stored on the cartridge via the barcode, which the instrument reads. In a preferred embodiment, the value of AALB is the independent variable input to the next linear regression performed on the sample and the pre-correction albumin concentration, C*_{A*LB} is returned as the dependent variable as shown in the following equation. C*_{ALB}=m·A_{ALB} b

In one example, once the pre-correction albumin concentration, C*_{ALB}, and the albumin dilution factor, DF_{ALB}, are known, the original albumin concentration was determined by correcting for the albumin dilution which occurs when the whole blood sample 605 is mixed with the reconstituted dyed agglutination reagent 612 and correcting for the 1:9 serial dilution of the original separated plasma drop 632 to produce the 1:9 albumin sample drop 656.

These corrections are shown in the following equation where [Albumin] is the original albumin concentration in the whole blood sample 605, C_{A}*_{LB} is the pre-correction albumin concentration determined from the standard curve using the background-corrected absorbance of the albumin-BCG complex, DF_{ALB} is the modeled dilution of the actual dilution experience by known albumin concentrations on the cartridge and the “9” corrects for the 1:9 dilution of the separated plasma drop 632.

[Albumin]=9×C*_{ALB}×DF_{ALB}

In some cases it may be helpful to establish methods and flags for colorimetric assays, such as the albumin assay. For example, it may be useful to use spectroscopic analysis to check that the dyed plasma 626 correctly separated from the whole blood sample 625. In one embodiment of the assay an absorbance measurement at 750 nm may be collected to confirm that the dyed plasma 626 correctly separated from the agglutinated whole blood 625.

In another example, it may be useful to check the dilution factors, e.g., for the albumin sample. In one aspect of the invention, the system may check apparent dilution factor upon a plasma sample going from approximately 1:3 to approximately 1:9. Plasma dilution may not be measured directly because of the unknown hematocrit ratio in each sample but may be determined indirectly by measuring the dilution of an added dye, such as Cy3 dye. The absorbance readings 615 of the 1:3 plasma drop 645 and the 1:9 plasma drop 655 may be used to calculate the actual dilution performed on the sample by comparing the absorbance measurements 615 of the two differently diluted drops. The 1:3 and 1:9 diluted plasma drops have important absorption 615 signals at 530 nm and 586 nm. Leveraging absorption 615 signals in this region may check for correct dilution.

In one aspect of the invention, it may be useful for the system to check whether the albumin sample droplet 656 absorbance measurement 615 shows a Cy3 dye concentration similar to that measured for the 1:9 waste drop 655. The 1DU albumin sample droplet 656 and the 2DU 1:9 waste droplet 655 are splits from the same drop. There could be subtle 1DU vs 2DU differences, but their absorbances 615 should be similar.

In another embodiment, the system may include flags and methods for determining whether there has been sufficient rehydration of the dyed PHAE drop 612. The rehydrated dyed PHAE drop 612 is colored and has important absorption signals 615 at 520 nm and 590 nm. Leveraging absorption 615 signals in this region may allow for qualification of rehydration success.

In another embodiment, the system includes flags and methods for determining whether there has been sufficient rehydration of the albumin reagent 660. Albumin reagent 660 is colored and has important absorption signals 615 at 510 nm and 750 nm. Leveraging absorption 615 signals in this region may allow for qualification of rehydration success.

In another embodiment, the system includes flags and methods for determining whether the albumin reagent 660 has been contaminated by dirt, dust or other types of physical contaminants. An albumin reagent 660 which is clean or contaminated by dirt or dust has important absorption signals 615 at 630 nm and 750 nm. Leveraging absorption 615 signals in this region may allow for qualification of the quality of the albumin reagent.

In some cases, it is useful to establish a flag in the method to ensure that the albumin reagent has not picked up high-protein material. For example, the flag may measure the absorbance 615 at 630-750 nm. The absorbance 615 of the albumin reagent 660 at 630 nm increases after it has encountered albumin (i.e. the point of the albumin assay). If the absorbance of the albumin reagent rises significantly before the reaction, it can be inferred that there is an interfering substance in the droplet. The absorbance at 750 nm may be subtracted from the absorbance at 630 nm to correct for small offsets of background noise.

Total Bilirubin Assay

As another example of a colorimetric assay, the inventors describe here a test for total bilirubin concentration using azobilirubin chemistry. The test may be performed on a droplet actuator using droplet operations. Understanding total bilirubin concentration is useful to inform a neonatal hyperbilirubinemia diagnosis and subsequent treatment based on American Academy of Pediatrics guidelines for hyperbilirubinemia. See American Academy of Pediatrics, Subcommittee on Hyperbilirubinemia, “Management of hyperbilirubinemia in the newborn infant 35 or more weeks of gestation,” Pediatrics 2004, 114, 297-316, which is incorporated herein by reference in its entirety.

The total bilirubin assay uses plasma as an input sample. Plasma may be separated from whole blood using a variety of techniques known in the art, such as centrifugation. In some cases it may be useful to first separate plasma from whole blood on the cartridge, as described above with respect to FIG. 5, before beginning the colorimetric assay.

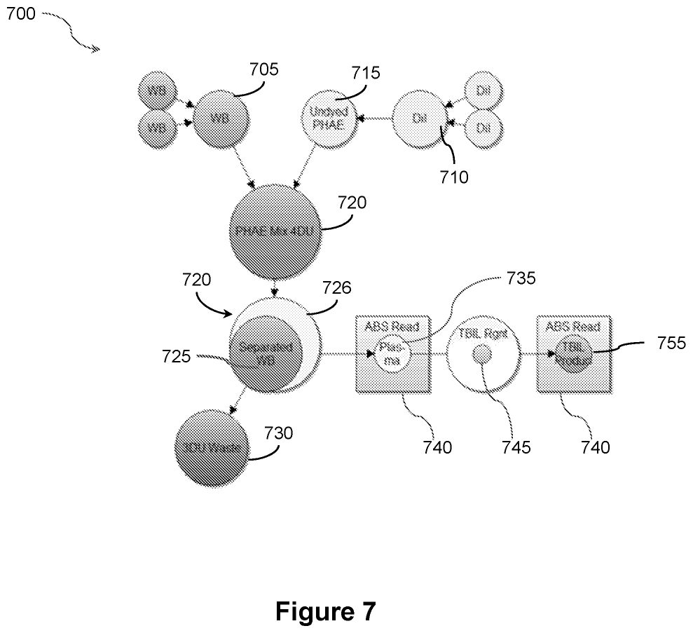

FIG. 7 illustrates a process for performing a total bilirubin assay according to the invention. Whole blood droplets can be dispensed from a whole blood sample (not shown) using droplet operations to provide an initial starting whole blood sample 705. As illustrated here, the whole blood sample 705 is a 2DU droplet.

Diluent droplets can be dispensed from a diluent source using droplet operations to provide an initial starting diluent aliquot droplet 710. As illustrated here, the diluent aliquot droplet 710 is a 2DU droplet.

The 2DU diluent aliquot droplet 710 may be transported using droplet operations into contact with a dried agglutination reagent (e.g., PHAE) to rehydrate the dried agglutination reagent and provide a reconstituted agglutination reagent droplet 715. The process of rehydrating the dried agglutination reagent may involve pulsing the droplet atop the agglutination reagent. Pulsing involves repeatedly activating and deactivating the underlying electrode. In some cases, the pulsing may involve at least 10 pulses. In other cases, at least 100 pulses.

In one aspect of the invention, the agglutination reagent does not include a dye or tracer, such as a fluorophore, because the dye or tracer interferes with other chemicals in the assay. For example, the Cy3 dye used in the albumin assay (described with reference to FIG. 6) interferes with the chemistry or colorimetry of the total bilirubin assay and therefore cannot be used in a similar manner to determine the amount of sample dilution. Therefore, in one aspect of the invention, the PHAE dilution factor determined for the albumin assay may be used to determine the PHAE dilution in the total bilirubin assay since the whole blood comes from the same sample and the droplet volumes are substantially the same.

A 2DU whole blood droplet 705 and the 2DU undyed reconstituted agglutination reagent droplet 715 may be merged using droplet operations to yield a 4DU agglutination droplet 720 to cause agglutination of the red blood cells. Upon agglutination of the red blood cells, the agglutination droplet 720 will separate into agglutinated red blood cells 725 and plasma 726.

Droplet operations may be employed to move the agglutination droplet 720 and thereby cause circulation within the agglutination droplet 720 to improve exposure of the red blood cells to the agglutination reagent. For example, as in the albumin assay performed by the inventors, the agglutination droplet 720 is transported using electrowetting-mediated droplet operations around a loop of 18-20 electrodes for at least 4.5 min at 500 ms steps, where the direction is reversed (clockwise/counterclockwise), for example, every 2 loops.

In order to separate out the plasma fraction from the agglutination droplet 720, it is useful to transport the agglutination droplet 720 in a consistent direction so that the frontmost portion of the agglutination droplet 720 will be lacking in agglutinated red blood cells and can be split off to provide a substantially clean plasma fraction. As the agglutination droplet 720 with agglutinated red blood cells 725 is transported in a single direction, the agglutinated red blood cells 725 tend to drift to the posterior portion of the droplet, while the anterior portion of the droplet remains substantially free of agglutinated red blood cells 725. Thus, by reversing transport directions, the invention provides for effective mixing of the agglutination reagent with the whole blood sample, and by maintaining a consistent direction, the invention provides for separation of the agglutinated red blood cells 725 from a portion of plasma 726. For example, as in the albumin assay (described above with reference to FIG. 6), following the mixing stage in which the direction around an electrode loop was changed, for example, every two times around the loop, the agglutinated droplet 720 may be transported around the loop in a consistent direction for six consecutive loops prior to splitting off the anterior portion of agglutinated droplet 720 to provide a 1DU plasma droplet 735 and a 3DU agglutinated red blood cell droplet 730 composed of the agglutinated red blood cells and a small fraction of unseparated plasma. The splitting step may be performed using a droplet splitting operation.

As in the albumin assay, there is an unknown dilution of the plasma due to unknown hematocrit per whole blood sample. The dilution of the plasma may be calculated in a manner similar to that in the albumin assay.

In one aspect, a total bilirubin dilution factor (DF_{TBIL}) may be calculated using the following experimentally determined equation. Because no dye or tracer is used in the agglutination reagent used in the total bilirubin assay, the dyed PHAE dilution factor (DF_{PHAE}) determined for the albumin assay may be used to determine the undyed PHAE dilution factor in the total bilirubin assay. The experimental method of determining the total bilirubin dilution factor model is as described for the experimentally determined albumin dilution factor above, where DF_{PHAE} was determined during the albumin assay. The model parameters for the total bilirubin assay are: α=438100, β=8.226 and γ=1.95.

DF_{TBIL}=αe^{(-β*DF}^{_{PHAE}}⁾+γ

Next, in one aspect of the invention, the absorbance 740 of the 1DU plasma droplet 735 may be measured once in the range of from about 450 nm to about 750 nm. In one aspect of the invention, the absorbance of an oil blank may be collected before and after the absorbance 740 of the 1DU plasma droplet 735. In a preferred aspect of the invention, the absorbance of an oil blank may be collected immediately before and immediately after the absorbance 740 of the 1DU plasma droplet 735. In one example, the absorbance measurement 740 of the 1DU plasma droplet 735 may be scatter corrected using a method similar to that described above with respect to the G6PD assay, where background scatter may be observed in the range of from about 650 nm to about 725 nm. With regard to the measurement of total bilirubin, the inventors have discovered that during detection at certain absorbance measurements relevant to total bilirubin, other compounds such as proteins in addition to the lipids discussed previously, cause light scattering, which confounds the total bilirubin signal. Consequently, the inventors have found it useful to correct for scatter by non-bilirubin compounds in order to achieve a more accurate determination of total bilirubin. By correcting for scatter caused by non-bilirubin compounds, the methods of the invention help to distinguish bilirubin absorption from non-bilirubin scatter, such as scatter from proteins or lipids. Similarly to that discussed in the G6PD assay, proteins cause an errant increase and a negative slope to the total bilirubin absorbance signal.

In one aspect of the invention, for a 1DU plasma droplet 735, a linear regression is performed on cartridge of measurements of the absorbance vs. wavelength for wavelengths of the range from about 650 nm to about 725 nm where optical scatter is predominantly present. In one aspect of the invention the simplified Rayleigh scatter model is extrapolated over the entire spectral range measured (from about 450 nm to about 750 nm) and used to fit the absorbance vs. wavelength data. In one aspect of the invention, protein scatter is removed by setting the exponent in the simplified Rayleigh scatter model to n=-1 such that the equation is now: A=+αλ⁻¹+c, where A is the absorbance of the sample and a and c are the linear regression fitting parameters, slope and intercept of a line, respectively. This simplified Rayleigh scattering model is then subtracted from the 1DU plasma droplet 735 absorbance 740 to obtain the protein scatter corrected absorbance of the undyed plasma sample.

After the absorbance measurement, the 1DU plasma droplet 735 is transported using droplet operations to a dried total bilirubin (TBIL) reagent spot 745. In one aspect of the invention the dried total bilirubin reagent is composed of 2 mM 3,5-Dichlorophenyldiazonium tetrafluoroborate, 2.0 mM dyphylline, 150 mM NaCI, and excipients in 50 mM maleate buffer at a pH of 3.2. The 1DU plasma droplet 735 is directly used to reconstitute the dried total bilirubin reagent spot 745 to yield a total bilirubin product droplet 755. The process of rehydrating the dried total bilirubin reagent may involve pulsing the 1DU plasma droplet 735 atop the bilirubin reagent spot 745. Pulsing involves repeatedly activating and deactivating the underlying electrode. In some cases, the pulsing may involve at least 10 pulses. In other cases, at least 100 pulses. In other cases, the pulsing may be followed by an incubation period. In other cases, pulsing may involve 30 seconds of pulsing (or approximately 30 pulses) followed by at least a 4 min incubation period.

Using plasma to rehydrate the total bilirubin reagent spot 745 eliminates the need to account for dilution as in the albumin assay. The benefit of this aspect of the invention is that it is much easier to quantify small changes in absorbance because there is no dilution of the reagent or sample.

In one example, after an incubation period, the absorbance 740 of the total bilirubin product droplet 755 may be measured over a desired spectral range. In another example, the absorbance 740 of the total bilirubin product droplet 755 may be measured once over the range of from about 450 nm to about 750 nm. In another example of the invention, the absorbance of an oil blank may be measured before and after the absorbance of 740 of the total bilirubin product droplet 755. In a preferred example, the absorbance of an oil blank may be measured immediately before and immediately after the absorbance 740 of the total bilirubin product droplet 755. Upon reconstitution and incubation with the total bilirubin reagent spot 745, bilirubin in the plasma droplet 735 transforms into azobilirubin, which causes a proportional increase in absorbance from that of the plasma and which has a concentration proportional to total bilirubin concentration in the sample. As described above, the absorbance spectrum of the total bilirubin product droplet 755 may be scatter corrected to remove signals from non-bilirubin compounds such as proteins, lipids, and oxyhemoglobin which is converted to methemoglobin during the bilirubin assay.

In one example, to isolate the azobilirubin absorbance signal from absorbance signals from non-bilirubin compounds, the scatter corrected absorbance of the plasma droplet 735 can be subtracted from the scatter corrected absorbance of the total bilirubin product droplet 755 at a chosen wavelength (A_{Azo}=A_{product}−Aₛₐₘₚₗₑ=A₇₅₅−A₇₃₅). In one aspect of the invention, the scatter corrected absorbance of the plasma droplet 735 measured at 519 nm is subtracted from the scatter corrected absorbance of the total bilirubin product droplet 755 measured at 519 nm resulting in a background signal corrected absorbance of the total bilirubin product droplet 755, A_{Azo}, which is due to azobilirubin. In one aspect of the invention, the linear scatter correction removes the protein signal from both the total bilirubin product droplet 755 and plasma droplet 735 absorbance spectra. In another aspect of the invention the interference due to lipids in the total bilirubin product droplet 755 negates the interference due to lipids in the plasma sample drop 735.

In another aspect of the invention, subtracting the absorbance of the plasma droplet 735 at 519 nm from the absorbance of the total bilirubin product droplet 755 at 519 nm negates the interference due to oxyhemoglobin in the plasma droplet 735, and from interference due to methemoglobin in the total bilirubin product droplet 755, both of which have similar extinction coefficients at 519 nm. The resulting value from the subtraction of the plasma droplet 735 absorbance from the total bilirubin product 755 absorbance spectra produces the background signal corrected absorbance of the azobilirubin in the total bilirubin product droplet 755, A_{Azo}.

In one aspect of the invention, the background signal corrected absorbance of the A_{Azo} may be converted to pre-corrected azobilirubin concentration using a factory generated standardized curve of pairs of known total bilirubin concentrations and the resultant azobilirubin absorbance value produced in the factory on the cartridge using the total bilirubin assay. For example, a linear regression may be experimentally determined in the factory from these data pairs and the linear regression slope and intercept values may be stored on the cartridge via the barcode, which the instrument reads. In one embodiment, AAzₒ values obtained on the cartridge may be multiplied by the stored slope and then that product may be added to the stored intercept to provide the pre-corrected albumin concentration. In one embodiment, when a new batch of a total bilirubin reagent is made, this calibration may be performed and the resulting linear regression parameters may be updated and then stored on the cartridge. In a preferred embodiment, the known TBIL concentration in the concentration-absorbance data sets above was first divided by the DF_{TBIL} determined for each sample on the cartridge. The linear regression was then performed using the following data pairs: [(known TBIL concentration/DF_{TBIL}), resultant azobilirubin absorbance values], as can be seen in the following equation, where Known [TBIL] and Absorbance of Known [Azo] are data pairs generated in the laboratory and DF_{TBIL} is generated for each sample run, where [TBIL] and [AZO] are the total bilirubin concentration and the azobilirubin concentration respectively.

$$\frac{Known [ TBIL ]}{{DF}_{TBIL}} = m \cdot Absorbance of Known [ AZO ] + b$$

In this preferred example, the linear regression was run for each sample and the slope and intercept values may be stored on the cartridge via the barcode, which the instrument reads. In a preferred embodiment, the value of A_{Azo} is the independent variable input to the linear regression performed on the sample and the pre-correction total bilirubin concentration, C*_{TBil} is returned as the dependent variable.

C*_{TBIL}=m·A_{Azo}b

In one embodiment of the total bilirubin assay, serial dilution of the sample was not performed; therefore further dilution corrections are not included. The original total bilirubin concentration in the whole blood sample 705, [Total Bilirubin], may be calculated using the following equation, where C*_{TBil} is the pre-correction total bilirubin concentration determined from the background-corrected absorbance of azobilirubin, DF_{TBIL} is the modeled dilution of the actual dilution experienced by known total bilirubin concentrations on the cartridge.

[Total Bilirubin]=C*_{TBil}×DF_{TBIL}

In some cases it may be helpful to establish methods and flags for colorimetric assays, such as the total bilirubin assay. For example, it may be useful to use spectroscopic analysis to check that the undyed plasma 726 correctly separated from the remainder of the whole blood 725. In one embodiment of the assay an absorbance measurement at about 750 nm may be collected to confirm that the undyed plasma 726 correctly separated from the agglutinated whole blood 725.

In another embodiment, the system includes flags and methods for determining whether there has been sufficient rehydration of the total bilirubin reagent 745. Total bilirubin present in the undyed plasma 726 is colored and has absorption signals around for example 500 nm. Absorption signals in this spectral region can be compared to assay-calculated [TBIL] to verify reconstitution of the dry reagent. In one aspect of the invention the total bilirubin absorption spectrum in plasma drop 726 may be scatter corrected using the non-linear method in the albumin assay and the wavelength range of about 700 nm to about 750 nm.

In some cases, it is useful to establish a flag in the bilirubin assay method to ensure that a plasma droplet does not show indications of hemolysis, which may have occurred, for example, from over handling of the whole blood sample. In one example, hemolysis in the plasma droplet 735 may be checked using an absorbance measurement 740 collected at about 577 nm after a non-linear scatter correction over the wavelength range of from about 700 nm to about 750 nm.

In some cases, it may be useful to check for lipemia in a plasma sample. In one example, an absorbance measurement 740 at about 700 nm may be used to assess a plasma droplet 735 for lipemia.

Other Quality Control Flags and Methods

The system of the invention includes a variety of quality-control methods and flags to ensure the integrity of the results. A flag may be a message communicated by the system to the user, such as for example, on a display screen, or via an audible sound. A flag may stop further processing of the assay. Alternatively, a flag may pause further processing of the assay and await user input before proceeding with the assay. The system may provide a user the opportunity to stop further processing of the assay following a flag. In other cases, the system may simply add an asterisk to the results, indicating the issue identified.

For example, the system may include quality control methods and flags for absorbance measurements. For example, reference droplets may be transported into the detection zone for absorbance measurements and the system may flag measurements having inconsistent results. Alternatively, absorbances may be taken from measurements without a droplet present, which is effectively a measurement of the oil. A flag for too much absorbance may determine if the detector is operating outside of its linear range; on the low end of absorbance, the flag may check to see if the detector is operating with too little signal, which risks noisy readings. Additionally, the absorbance measurement may provide an indicator of cartridge alignment, since a misaligned cartridge will not read correctly.

In some cases, inconsistent reference measurements result from droplets getting stuck under the detector. In other cases, inconsistent reference measurements may result from a misaligned cartridge. In some cases, the system may respond to an inconsistent reference read by instructing the user to check cartridge alignment. If the user confirms correct alignment of the cartridge, the system may identify the inconsistent reference measurement as a stuck droplet. In other embodiments, alignment of the cartridge may be checked with other sensors.

If the cartridge is inserted poorly enough to give an integration time above a certain threshold, this measurement may be flagged. Therefore a reading may be taken at a predetermined integration time and if the absorption does not exceed the threshold, then the system would conclude that alignment is insufficient. For example, in one aspect of the invention, if the cartridge is inserted poorly enough to give an integration time >10,000 microseconds, it will be flagged. For example, if a reading is taken at 10,000 microseconds integration time, and if the peak count does not exceed 14000, then the integration time found by the automatic integration time finder would be >10000 microseconds and the alignment would be flagged as insufficient. Higher integration times can stop the instrument from producing meaningful results, so the run should be restarted with a new cartridge.

In one aspect of the invention, it may be useful to include in the system flags a method for identifying fluorescent material in the fluorimeter path. For example, cartridges with a large background fluorescence measurement indicate possible dust contamination and threaten the performance of the fluorimeter. Flags indicating dust contamination may produce an error message indicating that the cartridge may not be used.

Various droplet operations can be checked using impedance detection, e.g., as described in Sturmer et al., U.S. Pat. No. 9,492,822, entitled “Microfluidic feedback using impedance detection,” issued on Nov. 15, 2016, the entire contents of which are incorporated herein by reference. For example, following a droplet splitting operation expected to produce two droplets, impedance can be used to determine whether two droplets were produced. In another example, following a droplet merging action expected to coalesce two droplets into one and then move it elsewhere, impedance can be used at the site of the merge to determine that the merge was successful and no droplet material was left behind. Similarly, following a droplet dispensing operation, impedance can be used to determine whether the expected number of dispensed droplets were produced. Moreover, after a droplet merging operation, impedance can be used to determine whether the droplets were merged as expected. Moreover, following loading of a sample or reagent into a droplet reservoir, impedance can be used to determine whether a sufficient amount of sample or reagent is present in the reservoir. Impedance can also be used to measure whether a sufficient amount of filler fluid is present in the droplet operations gap of the droplet actuator, e.g., following loading of the filler fluid. In each case, if there is insufficient reagent, or if droplet operations have not produced the expected results, or if there is insufficient filler fluid, a flag may indicate next steps, including for example terminating the run.

System firmware may also be monitored and return a flag when an aspect of the system is not working properly, e.g. at start up. For example, flags may be used to indicate whether a detector, heaters, power supply, temperature sensors, motors, such as deck motors or the HRM motor, and other firmware issues are present.

In various embodiments, pins connecting the electrodes can be tested to determine whether they are connected. In some cases, the pins may be tested to determine whether they produce suspiciously high or low values. A blank pin may be subtracted from the value of each pin, and each subtracted value may be compared to the no median subtracted value for that pin. In some cases, values checked may be the imaginary component of the waveform. To test connectivity, a low voltage may be applied to the pin and the resulting current waveform may be measured. In the event of a poor connection, the current is significantly lower than usual, allowing qualification of instrument-cartridge connectivity.

As discussed above, oil reads may be indicators of “background noise.” These oil reads are actually transmission detections used to calculate absorbance. That is, to calculate the absorbance of a sample droplet, the transmission (or “absorbance”, but what is technically being measured on a detector-level here is transmission) of the sample drop is measured and the transmission of the oil is measured. The two are compared to calculate the absorbance used in calculations. In some cases, a dark read correction may also be included in the calculations.

Additional Comments

The present invention may be implemented using hardware, software, or a combination thereof and may be implemented in one or more computer systems or other processing systems. In one aspect, the invention is directed toward one or more computer systems capable of carrying out the functionality described herein.

Various modifications and variations of the disclosed methods, compositions and uses of the invention will be apparent to the skilled person without departing from the scope and spirit of the invention. Although the invention has been disclosed in connection with specific preferred aspects or embodiments, it should be understood that the invention as claimed should not be unduly limited to such specific aspects or embodiments.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the presently disclosed subject matter.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

We claim:

1. A method of providing a reconstituted reagent, the method comprising:
(a) providing a dried reagent on a surface, wherein the dried reagent comprises one or more reagent components and a dye or tracer;
(b) contacting the dried reagent with a reconstitution buffer to provide a reconstituted reagent droplet;
(c) measuring the dye or tracer in the reconstituted reagent droplet to produce a dye or tracer measurement; and
(d) determining based on the dye or tracer measurement the extent of reconstitution of the dried reagent.

2. The method of claim 1 wherein the dried reagent comprises a buffer.

3. The method of claim 1 wherein the dried reagent comprises a diluent reagent.

4. The method of claim 1 wherein the dried reagent comprises an enzymatic substrate.

5. The method of claim 4 wherein the enzymatic substrate comprises NADP+.

6. The method of claim 4 wherein the enzymatic substrate comprises NADP+ and maleimide.

7. The method of claim 4 wherein the enzymatic substrate comprises G6P and NADP+.

8. The method of claim 1 wherein the dried reagent comprises an agglutination reagent.

9. The method of claim 8 wherein the agglutination reagent comprises a hemagglutinating carbohydrate binding protein.

10. The method of claim 8 wherein the agglutination reagent comprises a hemagglutinating lectin or isolectin or potato lectin.

11. The method of claim 8 wherein the agglutination reagent comprises phytohemagglutinin.

12. The method of claim 8 wherein the agglutination reagent comprises phytohemagglutinin-E (PHAE).

13. The method of claim 1 wherein the surface comprises a surface of a droplet actuator.

14. The method of claim 13 wherein the surface of the droplet actuator comprises a substrate surface in a droplet operations gap of the droplet actuator that is susceptible to contact by a droplet.

15. The method of claim 13 wherein the surface comprises a gap facing surface of a top substrate of the droplet actuator.

16. The method of claim 13 wherein the surface comprises a gap facing surface of a bottom substrate of the droplet actuator.

17. The method of claim 14 wherein the surface comprises a coating on a substrate surface in the droplet operations gap of the droplet actuator.

18. The method of claim 13 wherein the surface comprises a surface projecting into or otherwise facing a droplet operations gap of a droplet actuator.

19. The method of claim 1 wherein the dye or tracer is not a molecular label.

20. The method of claim 1 wherein the dye or tracer comprises a fluorophore.

21. The method of claim 20 wherein the dye or tracer comprises Cy3.

22. The method of claim 1 wherein the reconstitution buffer comprises a buffer that is substantially isotonic to blood.

23. The method of claim 22 wherein the reconstitution buffer comprises a phosphate buffer that is substantially isotonic to blood.

24. The method of claim 22 wherein the reconstitution buffer comprises a Tris buffer that is substantially isotonic to blood.

25. The method of claim 1 wherein the reconstitution buffer comprises a non-isotonic buffer that when combined with a reconstituted reagent is substantially isotonic to blood.

26. The method of claim 1 wherein measuring comprises:
(a) determining absorbance at a wavelength selected to measure the dye or tracer in the reconstituted reagent droplet, and
(b) producing a dye or tracer measurement based on the absorbance.

27. The method of claim 1 wherein measuring comprises:
(a) determining fluorescence at an excitation/emission wavelength selected based on the dye or tracer in the reconstitute reagent droplet; and
(b) and producing a dye or tracer measurement based on the fluorescence.

28. The method of claim 1 further comprising stopping an assay process if the extent of reconstitution of the dried reagent is not within a predetermined range.

29. The method of claim 1 further comprising adjusting an assay measurement based on the extent of reconstitution of the dried reagent.

30. The method of claim 1 further comprising using the dye or tracer measurement to create a dilution factor for a reconstituted reagent droplet that is further diluted in an assay process to provide a diluted reconstituted reagent.

31. The method of claim 30 further comprising assessing whether the diluted reconstituted reagent is within a predetermined range.

32. The method of claim 30 further comprising stopping an assay process if the diluted reconstituted reagent is not within a predetermined range.

33. A method of measuring hemoglobin in a sample, the method comprising:
(a) providing a sample droplet comprising hemoglobin;
(b) obtaining one or more absorbance measurements of the sample droplet at multiple wavelengths from a hemoglobin spectral range, thereby providing an uncorrected hemoglobin absorbance measurement(s);
(c) using a computing device, correct one or more absorbance measurements for scatter from non-hemoglobin compounds in the sample droplet, thereby providing a corrected hemoglobin absorbance measurement(s); and
(d) using a computing device, calculating based on the absorbance measurement(s) the amount of hemoglobin in the sample.

34. The method of claim 33 wherein the sample comprises non-lysed whole blood.

35. The method of claim 33 wherein the sample comprises partially lysed whole blood.

36. The method of claim 33 wherein the sample comprises lysed whole blood.

37. The method of claim 33 wherein obtaining absorbance measurements comprises determining absorbance of the sample droplet in a spectral range from about 400 nm to about 750 nm.

38. The method of claim 33 wherein obtaining absorbance measurements comprises determining absorbance of the sample droplet in a spectral range from about 540 nm to about 750 nm.

39. The method of claim 33 wherein correcting the absorbance measurements for non-hemoglobin compounds comprises subtracting the non-hemoglobin scatter absorbance measurement(s) from the hemoglobin absorbance measurement(s).

40. The method of claim 39 wherein the non-hemoglobin scatter absorbance measurement(s) are made at greater than about 650 nm.

41. The method of claim 39 wherein the non-hemoglobin scatter absorbance measurement(s) are made in a spectral range from about 700 nm to about 750 nm.

42. The method of claim 39 wherein the non-hemoglobin scatter absorbance measurement(s) are made at a wavelength selected to detect scatter from lipids.

43. The method of claim 33 wherein calculating the amount of hemoglobin in the sample comprises, using a computing device:
(a) obtaining one or more scatter-corrected absorbance measurements of the sample droplet at a wavelength where hemoglobin absorbs;
(b) multiplying by sample dilution factor;
(c) multiplying by the molar mass of hemoglobin;
(d) dividing by the optical path length; and
(e) dividing by the molar extinction coefficient at the wavelength where hemoglobin absorbs.

44. The method of claim 43 further comprising using the hemoglobin absorbance measurement(s) to assess a degree of red blood cell lysis in a sample droplet.

45. The method of claim 43 further comprising using the hemoglobin absorbance measurement(s) to assess the degree of red blood cell lysis in a sample droplet at two or more steps in an assay process.

46. The method of claim 43 further comprising stopping an assay process if the degree of red blood cell lysis in a sample droplet is not within a predetermined range.

47. A method of separating plasma from a whole blood sample on a droplet actuator, the method comprising:
(a) providing a sample droplet comprising whole blood;
(b) combining the sample droplet with an agglutination reagent droplet to provide a combined agglutination droplet comprising a plasma fraction and a red blood cell fraction;
(c) transporting the agglutination droplet in a consistent direction thereby producing a front section of the droplet lacking the red blood cell fraction; and
(d) promptly splitting the transported droplet to provide a plasma sample droplet and an agglutinated red blood cell droplet.

48. The method of claim 47 wherein separation of the plasma sample droplet from the agglutination droplet is accomplished without the use of beads.

49. The method of claim 47 wherein separation of the plasma sample droplet from the agglutination droplet is accomplished without the use of magnetically responsive beads or magnets.

50. The method of claim 47 wherein the splitting is accomplished in a droplet operations gap of a droplet actuator using an electrowetting mediated splitting operation.

51. The method of claim 47 wherein the splitting is accomplished in a droplet operations gap of a droplet actuator using an electrowetting mediated splitting operation without the use of magnetically responsive beads or magnets.

52. The method of claim 47 further comprising providing a plasma sample droplet for a colorimetric dye-binding assay.

53. The method of claim 47 further comprising providing a plasma sample droplet for a total bilirubin and/or an albumin assay.

54. A method of measuring albumin in a plasma sample, the method comprising:
(a) providing a plasma droplet, wherein the plasma droplet comprises a dye or tracer reagent;
(b) measuring the dye or tracer in the plasma droplet to produce a plasma dye or tracer measurement, thereby providing a baseline plasma dilution factor;
(c) diluting the plasma droplet with a diluent to provide a diluted plasma droplet and a waste plasma droplet wherein the diluted plasma droplet and the waste plasma droplet comprise the dye or tracer reagent;
(d) measuring the dye or tracer in the diluted plasma droplet to produce a diluted plasma dye or tracer measurement, thereby providing a measurement for assessing the dilution process of the plasma from a whole blood sample;
(e) combining the diluted plasma droplet with a rehydrated albumin reagent droplet to provide an albumin product droplet comprising an albumin-reagent complex;
(f) measuring the absorbance of the albumin-reagent complex in the albumin product droplet, thereby providing a measurement for assessing the amount of albumin in the albumin product droplet;
(g) using a computing device, correcting the albumin-reagent complex absorbance measurement for background interfering signals, thereby providing a background corrected albumin-reagent complex absorbance measurement (A_{ALB});
(h) using a computing device, calculating based on the background corrected albumin-reagent complex absorbance measurement (A_{ALB}) the amount of albumin in the diluted plasma sample (C*_{ALB}); and
(i) using a computing device, calculating based on the amount of albumin in the diluted plasma droplet (C*_{ALB}) the amount of albumin in a corresponding whole blood sample.

55. The method of claim 54 wherein providing a plasma droplet from a whole blood sample comprises the method of claim 47.

56. The method of claim 54 wherein the dye or tracer is not a molecular label.

57. The method of claim 54 wherein the dye or tracer comprises a fluorophore.

58. The method of claim 57 wherein the dye or tracer comprises Cy3.

59. The method of claim 54 wherein measuring the dye or tracer in the plasma droplet to provide a baseline plasma dilution factor (DF_{PHAE}) comprises:
(a) obtaining an absorbance spectra of the plasma droplet over a spectral range of from about 650 nm to about 725 nm;
(b) obtaining an absorbance spectra of a reference dye or tracer sample over a spectral range of from about 650 nm to about 725 nm;
(c) using a computing device, correcting the plasma droplet absorbance spectra and the reference dye or tracer absorbance spectra for scatter or background absorbance over a spectral range of from about 650 nm to about 750 nm;
(d) using a computing device, calculating a scatter-corrected average absorbance spectrum for the plasma droplet and a scatter-corrected average absorbance spectrum for the reference dye or tracer sample;
(e) using a computing device, correcting the scatter-corrected average absorbance spectrum for the plasma droplet and the scatter-corrected average absorbance spectrum for the reference dye or tracer sample for hemoglobin absorbance; and
(f) using a computing device, calculating the baseline plasma dilution factor (DF_{PHAE}) as the ratio of hemoglobin-corrected reference dye or tracer absorbance to hemoglobin-corrected plasma droplet absorbance.

60. The method of claim 59 wherein the absorbance spectra of the plasma droplet and the absorbance spectra of the reference dye or tracer sample are obtained two or more times.

61. The method of claim 59 wherein the absorbance spectra of the plasma droplet and the absorbance spectra of the reference dye or tracer sample are obtained four times.

62. The method of claim 59 further comprising obtaining an absorbance measurement from about 650 nm to about 725 nm of an oil filler fluid blank before and after each spectral measurement of the plasma droplet and the reference dye or tracer sample, thereby providing a measure of background noise.

63. The method of claim 59 wherein the dye or tracer comprises Cy3.

64. The method of claim 59 wherein the correcting the scatter-corrected average absorbance spectra for hemoglobin absorbance comprises subtracting absorbance measurements at about 583 nm from absorbance measurements at about 556 nm, thereby providing an absorbance measurements for Cy3 in the plasma droplet and the reference dye or tracer sample.

65. The method of claim 54 wherein diluting the plasma droplet comprises serially diluting the plasma droplet.

66. The method of claim 65 wherein serially diluting the plasma droplet comprises a series of 1:3 dilutions.

67. The method of claim 66 wherein the 1:3 dilution is performed two times.

68. The method of claim 54 wherein measuring the dye or tracer in the diluted plasma droplet to provide a measurement for assessing the dilution process comprises:
(a) obtaining an absorbance measurement of a plasma droplet from each step in the dilution series;
(b) comparing the absorbance measurements from each plasma droplet in the dilution series;
(c) using a computing device, calculating based on the absorbance measurements from each plasma droplet the actual dilution performed at each step in the dilution process.

69. The method of claim 68 wherein the absorbance measurements of the dye or tracer in the diluted plasma droplets are obtained at about 556 nm and at about 583 nm.

70. The method of claim 68 wherein the absorbance measurements of the diluted plasma droplets are obtained one time.

71. The method of claim 68 further comprising obtaining an absorbance measurement of an oil filler fluid blank before and after the absorbance measurement of the diluted plasma droplet, thereby providing a measure of background noise.

72. The method of claim 71 wherein the absorbance measurement of the oil filler fluid blank is performed over the wavelengths from about 480 nm to about 775 nm.

73. The method of claim 54 wherein combining the diluted plasma droplet with an albumin reagent droplet comprises:
(a) obtaining a diluted plasma droplet absorbance measurement at a wavelength selected to remove background noise signals prior to combining the diluted plasma droplet with the albumin reagent droplet, thereby providing a background diluted plasma droplet measurement; and
(b) obtaining an albumin reagent droplet absorbance measurement at a wavelength selected to remove background noise signals prior to combining the albumin reagent droplet with the diluted plasma droplet, thereby providing a background albumin reagent droplet measurement.

74. The method of claim 73 wherein the absorbance measurement to remove background signals is performed at about 630 nm.

75. The method of claim 54 wherein the albumin reagent droplet comprises an albumin-binding dye and a buffer.

76. The method of claim 75 wherein the albumin-binding dye comprises a triphenylmethane dye.

77. The method of claim 76 wherein the triphenylmethane dye comprises bromocresol green.

78. The method of claim 75 wherein the buffer comprises a surfactant, a detergent, and a cyclodextrin.

79. The method of claim 78 wherein the surfactant comprises a zwitterionic surfactant.

80. The method of claim 79 wherein the zwitterionic surfactant comprises CHAPS.

81. The method of claim 78 wherein the detergent comprises a Brij® surfactant.

82. The method of claim 78 wherein the cyclodextrin comprises methyl-beta-cyclodextrin.

83. The method of claim 54 wherein measuring the absorbance of the albumin-reagent complex in the albumin product droplet comprises:
(a) obtaining an absorbance measurement of the albumin-reagent complex at a wavelength selected to detect absorption from the albumin-binding dye;
(b) obtaining an absorbance measurement of an oil filler fluid blank before and after the absorbance measurement of the albumin-reagent complex, thereby providing a measure of background noise; and
(c) subtracting the oil filler fluid absorbance measurement from the albumin-reagent complex absorbance measurement, thereby providing a noise corrected albumin-reagent complex measurement.

84. The method of claim 83 wherein the absorbance measurements of the albumin-reagent complex and the oil filler fluid blank is obtained at about 630 nm.

85. The method of claim 54 wherein correcting the albumin-reagent complex absorbance measurement for interfering signals comprises:
(a) multiplying the background diluted plasma droplet measurement by 0.5, thereby providing a dilution corrected diluted plasma droplet measurement;
(b) multiplying the background albumin reagent droplet measurement by 0.5, thereby providing a dilution corrected background albumin reagent droplet measurement; and
(c) subtracting the dilution corrected background albumin reagent droplet measurement and the dilution corrected diluted plasma droplet measurement from the noise corrected albumin-reagent complex measurement, thereby providing a corrected albumin-reagent complex absorbance measurement (A_{ALB}).

86. The method of claim 54 wherein calculating the amount of albumin in the diluted plasma sample comprises, using a computing device:
(a) providing a data set of known albumin concentrations and corresponding absorbance values;
(b) using a computing device, correcting the data set to provide a data set with adjusted known albumin concentrations;
(c) performing a linear regression analysis using the corrected data set of known albumin concentrations and their corresponding absorbance values to generate a standard curve; and
(d) using the corrected albumin-reagent complex absorbance measurement (A_{ALB}) and the linear regression slope and intercept values of the standard curve to calculate the amount of albumin in the diluted plasma droplet (C*_{ALB}).

87. The method of claim 86 wherein correcting the data set comprises, using a computing device:
(a) providing a final albumin dilution factor (DF_{ALB});
(b) providing a final serial dilution factor; and
(c) dividing the known albumin concentrations in the data set by the product of the final albumin dilution factor and the final serial dilution factor, thereby providing a data set with adjusted known albumin concentrations.

88. The method of claim 87 wherein providing the final albumin dilution factor (DF_{ALB}) comprises:
(a) providing a data set of known albumin sample concentrations and hematocrit concentrations;
(b) modeling the data set to determine best fit parameters; and
(c) using a computing device, calculating the final albumin dilution factor (DF_{ALB}) based on the best fit parameters and the baseline plasma dilution factor (DF_{PHAE}).

89. The method of claim 87 wherein providing a final serial dilution factor comprises determining the final dilution.

90. The method of claim 54 wherein calculating the amount of albumin in the corresponding whole blood sample comprises, using a computing device:
(a) multiplying the amount of albumin in the diluted plasma droplet (C*_{ALB}) by the final albumin dilution factor (DF_{ALB}) and thereby providing a plasma albumin concentration; and
(b) multiplying the plasma albumin concentration by the final serial dilution factor to correct for the serial dilution of the plasma sample, thereby providing a measurement of the amount of albumin in the whole blood sample.

91. The method of claim 86 wherein the data set of known albumin concentrations and corresponding absorbance values are stored on cartridge.

92. The method of claim 86 wherein the linear regression slope and intercept values are stored on cartridge.

93. The method of claim 92 wherein the stored linear regression slope and intercept values are accessed via reading a barcode.

94. The method of claim 54 further comprising stopping the assay process if the dilution of a plasma droplet is not within a predetermined range.

95. The method of claim 54 further comprising stopping the assay process if an absorbance measurement of a diluted plasma droplet and the corresponding waste plasma droplet are not within a predetermined range.

96. The method of claim 54 further comprising stopping the assay process if the rehydrated albumin reagent is not sufficiently rehydrated.

97. The method of claim 96 wherein determining whether the albumin reagent has been sufficiently rehydrated comprises:
(a) measuring the absorbance of the rehydrated albumin reagent at about 510 nm and about 750 nm; and
(b) determining based on the absorbance measurements whether the albumin reagent has been sufficiently rehydrated.

98. The method of claim 54 further comprising stopping the assay process if the rehydrated albumin reagent is contaminated by a physical contaminant.

99. The method of claim 98 wherein determining whether the rehydrated albumin reagent is contaminated by a physical contaminant comprises:
(a) measuring the absorbance of the rehydrated albumin reagent at about 630 nm and about 750 nm; and
(b) determining based on the absorbance measurements if the quality of rehydration of the albumin reagent is within a predetermined range.

100. The method of claim 54 further comprising stopping the assay process if the rehydrated albumin reagent is contaminated by a high-protein material.

101. The method of claim 100 wherein determining whether the rehydrated albumin reagent is contaminated by a high-protein material comprises:
(a) measuring the absorbance of the rehydrated albumin reagent at about 630 nm and about 750 nm prior to combining the albumin reagent droplet with the diluted plasma droplet;
(b) comparing the absorbance of the rehydrated albumin reagent to the typical absorbance of the rehydrated albumin reagent at these wavelengths;
(c) determining if the absorbance of the rehydrated albumin reagent is higher relative to the typical absorbance of the rehydrated albumin reagent at these wavelengths;
(d) calling the rehydrated albumin reagent as contaminated by a high-protein material if the absorbance of the rehydrated albumin reagent is higher than the typical absorbance of the rehydrated albumin reagent at these wavelengths; and
(e) using a computing device, correct the absorbance measurement of the rehydrated albumin reagent to offset background noise by subtracting the absorbance measurement at about 750 nm from the absorbance measurement at about 630 nm.

102. A method of measuring G6PD activity in a sample, the method comprising:
(a) providing a sample droplet comprising whole blood;
(b) combining the sample droplet with a G6PD stabilizing reagent droplet to provide a G6PD stabilized sample droplet;
(c) obtaining an absorbance measurement of hemoglobin in the combined reagent and sample droplet, thereby providing a G6PD stabilized sample droplet measure of red blood cell lysis;
(d) diluting the combined reagent and sample droplet with a diluent to provide a lysed sample droplet;
(e) obtaining an absorbance measurement of hemoglobin in the lysed sample droplet to provide a lysed sample droplet measure of:
(i) red blood cell lysis; and
(ii) an apparent dilution factor;
combining the lysed sample droplet with a G6PD substrate reagent droplet to provide a G6PD−-substrate reaction droplet;
(g) detecting a reaction product in the G6PD−-substrate reaction droplet to produce a measurement of G6PD activity in the sample; and
(h) converting the measure of G6PD activity in the sample to a final assay output of a measure of G6PD activity per grams of hemoglobin (U/g Hb).

103. The method of claim 102 wherein the G6PD stabilizing reagent comprises NADP+ and maleimide.

104. The method of claim 102 wherein obtaining an absorbance measurement of hemoglobin to provide a G6PD stabilized sample droplet measure of red blood cell lysis comprises:

105. obtaining one or more absorbance measurements of the G6PD stabilized sample droplet at multiple wavelengths from a hemoglobin spectral range, thereby providing an uncorrected hemoglobin absorbance measurement(s); and

106. using a computing device, correct one or more absorbance measurements for scatter from non-hemoglobin compounds in the G6PD stabilized sample droplet, thereby providing a corrected hemoglobin absorbance measurement and a measure of red blood cell lysis.

107. The method of claim 104 wherein obtaining absorbance measurements comprises determining absorbance of the G6PD stabilized sample droplet in a spectral range from about 540 nm to about 750 nm.

108. The method of claim 104 wherein correcting one or more absorbance measurements for scatter from non-hemoglobin compounds in the G6PD stabilized sample droplet comprises subtracting the hemoglobin absorbance measurement at about 750 nm from the hemoglobin absorbance measurement at about 540 nm.

109. The method of claim 102 wherein diluting the combined reagent and sample droplet comprises serially diluting the combined reagent and sample droplet.

110. The method of claim 107 wherein serially diluting the combined reagent and sample droplet comprises a series of 1:3 dilutions.

111. The method of claim 108 wherein the 1:3 dilution is performed two times.

112. The method of claim 102 wherein obtaining an absorbance measurement of hemoglobin in the lysed sample droplet comprises:
(a) obtaining one or more absorbance measurements of the lysed sample droplet at multiple wavelengths from a hemoglobin spectral range, thereby providing an uncorrected hemoglobin absorbance measurement(s);
(b) using a computing device, correct one or more absorbance measurements for scatter from non-hemoglobin compounds in the lysed sample droplet to provide a corrected hemoglobin absorbance measurement, thereby providing measure of red blood cell lysis and an apparent dilution factor.

113. The method of claim 110 wherein obtaining absorbance measurements comprises determining absorbance of the lysed sample droplet in a spectral range from about 540 nm to about 750 nm.

114. The method of claim 110 wherein correcting one or more absorbance measurements for scatter from non-hemoglobin compounds in the lysed sample droplet comprises subtracting the hemoglobin absorbance measurement at about 750 nm from the hemoglobin absorbance measurement at about 540 nm.

115. The method of claim 110 further comprising stopping the assay process if the measure of red blood cell lysis of the sample droplet is not within a predetermined range.

116. The method of claim 110 wherein the measure of the apparent dilution factor comprises comparing the absorbance measurement of each lysed sample droplet in the dilution series obtained at 540 nm, thereby providing a measure of apparent dilution.

117. The method of claim 110 further comprising stopping the assay process if the apparent dilution factor of the sample droplet is not within a predetermined range.

118. The method of claim 102 wherein the G6PD substrate reagent droplet comprises G6P and NADP+

119. The method of claim 102 wherein detecting the product of the G6PD−-substrate reaction comprises obtaining kinetic fluorescence measurements of NADPH in the G6PD−-substrate reaction droplet.

120. The method of claim 117 wherein the kinetic fluorescence measurements of NADPH are obtained in a fluorescence channel from about 360 nm to about 460 nm.

121. The method of claim 117 wherein the kinetic fluorescence measurements are obtained at about 10 second intervals.

122. The method of claim 117 wherein obtaining kinetic fluorescence measurements comprises performing one or more cycles of kinetic fluorescence measurements, thereby providing a set of kinetic fluorescence measurements.

123. The method of claim 120 wherein the set of kinetic fluorescence measurements comprises about 5 cycles or about 10 cycles or about 15 cycles or about 20 cycles or about 25 cycles or more.

124. The method of claim 117 wherein obtaining kinetic fluorescence measurements is performed in less than about 5 minutes.

125. The method of claim 117 wherein obtaining kinetic fluorescence measurements is performed in less than about 4 minutes.

126. The method of claim 117 further comprising using a sweeper buffer droplet between fluorescence measurements to substantially remove or reduce any remnants of the G6PD−-substrate reaction droplet prior to obtaining a subsequent fluorescence measurement.

127. The method of claim 102 wherein detecting the product of the G6PD−-substrate reaction to produce the measurement of G6PD activity comprises:
(a) obtaining a fluorescence measurement of an oil filler fluid blank before and after obtaining a fluorescence measurement of the G6PD−-substrate reaction droplet, thereby providing a measure of background noise;
(b) averaging the fluorescence measurements of the oil filler fluid blank obtained before and after the fluorescence measurement of the G6PD−-substrate reaction droplet, thereby providing an average blank relative fluorescence unit (RFU) value;
(c) subtracting the average blank RFU value from the fluorescence measurement of the G6PD−-substrate reaction droplet, thereby providing a list of subtracted RFU data;
(d) plotting the subtracted RFU data vs time;
(e) applying a weighting function to the data to remove outlying data points;
using a computing device, calculating a slope, intercept, and a R² linear regression parameter, thereby providing a measure of NADPH RFU per minute (NADPH RFU/min);
(g) converting the NADPH RFU/min value to a measure of pM NADPH production units of activity per minute (pM NADPH/min); and
(h) converting the measure of NADPH production units of activity to activity units per liter (U/L), thereby providing a measure G6PD activity per liter (G6PD U/L).

128. The method of claim 125 wherein the weighting function comprises:
(a) subjecting positive residuals to a weighting of (1/residual), thereby minimizing their influence on a best fit line;
(b) capping the weighting of very small residuals at a given maximum to prevent infinitely high weights, while maximizing their influence on the best fit line; and
(c) subjecting negative residuals to a weighting that is between (1/residual) and the maximum small-residual weight (inclusive).

129. The method of claim 125 wherein calculating the fit of the linear regression parameter is an iterative process, thereby providing the best least absolute residual (LAR) fit.

130. The method of claim 125 further comprising stopping the assay process if a good regression fit cannot be identified.

131. The method of claim 128 wherein identifying a good regression fit comprises, using a computing device:
(a) calculating the residuals for each data point from the best-fit curve;
(b) marking as poorly fit residuals that exceed:
(i) a flat allowable error; and
(ii) a percent-based allowable error; and
(c) flagging the fit as bad if the number of poorly fit points exceeds a predetermined value.

132. The method of claim 129 wherein the flat allowable error comprises 2RFU.

133. The method of claim 129 wherein the percent-based allowable error is about 20%.

134. The method of claim 102 wherein the measure of G6PD activity is converted to G6PD activity per grams of hemoglobin comprises, using a computing device:
(a) calculating the amount of hemoglobin in the G6PD−-substrate reaction droplet, thereby providing a measure of the amount of hemoglobin in the diluted reaction droplet;
(b) multiplying the hemoglobin concentration in the diluted reaction droplet by a final dilution factor, thereby providing a measure of the hemoglobin concentration (g Hb/L) in the whole blood sample; and
(c) dividing the measure of G6PD activity (G6PD U/L) by the measure of the hemoglobin (g Hb/L) in the whole blood sample; thereby providing the measure of G6PD activity per grams of hemoglobin (G6PD U/g Hb).

135. The method of claim 132 wherein calculating the amount of hemoglobin in the G6PD−-substrate reaction droplet comprises, using a computing device:
(a) providing a sample droplet comprising hemoglobin;
(b) obtaining one or more absorbance measurements of the sample droplet at multiple wavelengths from a hemoglobin spectral range, thereby providing an uncorrected hemoglobin absorbance measurement(s);
(c) using a computing device, correct one or more absorbance measurements for scatter from non-hemoglobin compounds in the sample droplet, thereby providing a corrected hemoglobin absorbance measurement(s); and
(d) calculating based on the absorbance measurement(s) the amount of hemoglobin in the sample.

136. The method of claim 133 wherein the absorbance value for calculating the amount of hemoglobin is obtained at about 524 nm.

137. The method of claim 132 wherein the final dilution factor is 54x.

138. The method of claim 132 further comprising stopping the assay process if the amount of hemoglobin is not within a predetermined range.

139. A method of measuring total bilirubin in a plasma sample, the method comprising:
(a) providing a plasma droplet wherein the plasma droplet does not comprise a dye or tracer reagent;
(b) obtaining absorbance measurement of the plasma droplet over a spectral range, thereby providing a background plasma droplet absorbance measurement;
(c) using a computing device, correct the background plasma droplet absorbance measurement for scatter by non-bilirubin compounds, thereby providing a scatter corrected background plasma droplet absorbance measurement;
(d) contacting a dried total bilirubin reagent with the plasma droplet wherein the plasma droplet rehydrates the total bilirubin reagent to provide a reaction droplet comprising a total bilirubin product;
(e) obtaining absorbance measurements of the total bilirubin product in the reaction droplet over a spectral range, thereby providing an absorbance measurement for assessing the amount of total bilirubin in the reaction droplet;
(f) using a computing device, correct the absorbance measurement of the total bilirubin product for scatter by non-bilirubin compounds, thereby providing a scatter corrected total bilirubin product absorbance measurement;
(g) isolating an absorbance signal at a selected wavelength for the total bilirubin product in the reaction droplet, thereby providing an isolated absorbance measurement of the total bilirubin product in the reaction droplet (A_{AZO}); and
(h) using a computing device, calculating based on the isolated absorbance measurement of the total bilirubin product in the reaction droplet (A_{AZO}) the amount of total bilirubin in the plasma sample (C*_{TBIL});
(i) using a computing device, calculating based on the amount of total bilirubin in the plasma droplet (C*_{TBIL}) the amount of total bilirubin in a corresponding whole blood sample.

140. The method of claim 137 wherein providing a plasma droplet from a whole blood sample comprises the method of claim 47.

141. The method of claim 137 wherein providing a background plasma droplet absorbance measurement comprises:
(a) obtaining an absorbance measurement of the plasma droplet over a spectral range, thereby providing an uncorrected background plasma droplet absorbance measurement;
(b) obtaining an absorbance measurement of an oil filler fluid blank over the same spectral range, thereby providing a measure of background noise; and
(c) subtracting the oil filler fluid absorbance measurement from the uncorrected background plasma droplet absorbance measurement, thereby providing a noise corrected background plasma droplet absorbance measurement.

142. The method of claim 139 wherein the absorbance measurement of the plasma droplet and the oil filler fluid blank are obtained at from about 450 nm to about 750 nm.

143. The method of claim 139 wherein the absorbance measurement of the plasma droplet is obtained once.

144. The method of claim 139 wherein the absorbance measurements of the oil filler fluid blank is obtained before and after the absorbance measurement of the plasma droplet.

145. The method of claim 137 wherein correcting the background plasma droplet absorbance measurement for scatter by non-bilirubin compounds comprises subtracting non-bilirubin scatter absorbance measurements from the background plasma droplet absorbance measurement.

146. The method of claim 143 wherein the non-bilirubin scatter absorbance measurements are made over the spectral range from about 450 nm to about 750 nm.

147. The method of claim 143 wherein the non-bilirubin scatter absorbance measurements are made over the spectral range from about 650 nm to about 725 nm.

148. The method of claim 143 wherein the non-bilirubin scatter absorbance measurements are made at a wavelength selected to detect scatter from proteins.

149. The method of claim 143 wherein the non-bilirubin scatter absorbance measurements are made at a wavelength selected to detect scatter from lipids.

150. The method of claim 137 wherein the total bilirubin reagent comprises the azobilirubin chemistry reagents 3, 5-Dichlorophenyldiazonium tetrafluoroborate and dyphylline.

151. The method of claim 137 wherein providing an absorbance measurement for assessing the amount of total bilirubin product in the reaction droplet comprises:
(a) obtaining an absorbance measurement of the total bilirubin product in the reaction droplet over a spectral range, thereby providing an uncorrected total bilirubin product absorbance measurement;
(b) obtaining an absorbance measurement of an oil filler fluid blank over the same spectral range, thereby providing a measure of background noise; and
(c) subtracting the oil filler fluid absorbance measurement from the uncorrected total bilirubin product absorbance measurement, thereby providing a noise corrected total bilirubin product absorbance measurement.

152. The method of claim 149 wherein the absorbance measurement of the total bilirubin product and the oil filler fluid blank are obtained at from about 450 nm to about 750 nm.

153. The method of claim 149 wherein the absorbance measurement of the total bilirubin product in the total bilirubin reaction droplet is obtained once.

154. The method of claim 149 wherein the absorbance measurements of the oil filler fluid blank is obtained before and after the absorbance measurement of the total bilirubin product in the reaction droplet.

155. The method of claim 137 wherein correcting the total bilirubin product absorbance measurement for scatter by non-bilirubin compounds comprises subtracting non-bilirubin scatter absorbance measurements from the total bilirubin product absorbance measurement.

156. The method of claim 153 wherein the non-bilirubin scatter absorbance measurements are made over the spectral range from about 450 nm to about 750 nm.

157. The method of claim 153 wherein the non-bilirubin scatter absorbance measurements are made over the spectral range from about 650 nm to about 725 nm.

158. The method of claim 153 wherein the non-bilirubin scatter absorbance measurements are made at a wavelength selected to detect scatter from proteins.

159. The method of claim 153 wherein the non-bilirubin scatter absorbance measurements are made at a wavelength selected to detect scatter from lipids.

160. The method of claim 137 wherein isolating the absorbance signal of the total bilirubin product at a selected wavelength comprises subtracting the scatter corrected background plasma droplet absorbance measurement from the scatter corrected total bilirubin product absorbance measurement and thereby providing an isolated absorbance signal for the total bilirubin product (A_{AZO}).

161. The method of claim 158 wherein the isolated absorbance signal is obtained at about 519 nm.

162. The method of claim 137 wherein calculating the amount of total bilirubin in the plasma sample (C*_{TBIL}) comprises:
(a) providing a data set of known total bilirubin concentrations and corresponding azobilirubin absorbance values;
(b) using a computing device, correct the data set to provide a data set with adjusted known total bilirubin concentrations;
(c) performing a linear regression analysis using the corrected data set of know total bilirubin concentrations and their corresponding absorbance values to generate a standard curve; and
(d) using the total bilirubin product (A_{AZO}) isolated absorbance signal and the linear regression slope and intercept values of the standard curve to calculate the amount of total bilirubin in the plasma droplet (C*_{TBIL}).

163. The method of claim 160 wherein correcting the data set comprises, using a computing device:
(a) providing a final total bilirubin dilution factor (DF_{TBIL}); and
(b) dividing the known total bilirubin concentrations in the data set by the final total bilirubin dilution factor, thereby providing a data set with adjusted known total bilirubin concentrations.

164. The method of claim 161 wherein providing the final total bilirubin dilution factor (DF_{TBIL}) comprises, using a computing device:
(a) providing a data set known total bilirubin sample concentrations and hematocrit concentrations;
(b) modeling the data set to determine best fit parameters; and
(c) calculating the final total bilirubin dilution factor (DF_{TBIL}) based on the best fit parameters and a baseline plasma dilution factor.

165. The method of claim 162 wherein the baseline plasma dilution factor comprises a baseline dilution factor (DF_{PHAE}) obtained for a plasma droplet in an albumin assay performed using the method of claim 54.

166. The method of claim 160 wherein the data set of known total bilirubin concentrations and corresponding azobilirubin absorbance values are stored on cartridge.

167. The method of claim 160 wherein the linear regression slope and intercept values are stored on cartridge.

168. The method of claim 165 wherein the stored linear regression slope and intercept values are accessed via reading a barcode.

169. The method of claim 162 wherein calculating the amount of total bilirubin in the corresponding whole blood sample comprises multiplying the amount of total bilirubin in the plasma droplet (C*_{TBIL}) by the final total bilirubin dilution factor (DF_{TBIL}) and thereby providing a measurement of the amount of albumin in the whole blood sample.

170. The method of claim 169 further comprising obtaining an absorbance measurement of the plasma droplet at about 750 nm, thereby providing a measure for assessing the quality of the plasma droplet.

171. The method of claim 168 further comprising stopping the assay process of the absorbance measurement is not within a predetermined range.

172. The method of claim 168 further comprising stopping the assay process if the rehydrated total bilirubin reagent is not sufficiently rehydrated.

173. The method of claim 170 wherein determining whether the total bilirubin reagent has been sufficiently rehydrated comprises:
(a) measuring the absorbance of the plasma sample at about 498 nm and at about 590 nm; and
(b) determining based on the absorbance measurements whether the sample contains substantial total bilirubin; and
(c) comparing the TBIL assay result to the presence of substantial total bilirubin visible in the plasma sample to determine whether the total bilirubin reagent has been sufficiently rehydrated.

174. The method of claim 137 further comprising stopping the assay process if the plasma droplet shows indications of hemolysis.

175. The method of claim 172 wherein determining if the plasma droplet shows indications of hemolysis comprises:
(a) obtaining an absorbance measurement obtained at about 577 nm; and
(b) using a computing device, correcting the absorbance measurement of the plasma drop for scatter, thereby providing a scatter corrected plasma drop absorbance measurement; and
(c) determining based on the absorbance measurement whether the plasma droplet shows signs of hemolysis.
(d) The method of claim 137 further comprising screening for lipemia in the plasma sample.

176. The method of claim 174 wherein screening for lipemia comprises obtaining an absorbance measurement of the plasma droplet at about 750 nm, thereby providing a measure for assessing the plasma sample for lipemia.

177. The method of claim 47 further comprising performing a measurement of an analyte in the plasma sample droplet.
